(19) United States
(12) Patent Application Publication
(10) Pub. No.: US 2026/0263402 A1
(43) Pub. Date: Sep. 10, 2026

(54) SYNERGISTIC COMBINATIONS OF REGULATED NECROSIS INHIBITORS WITH N-ACETYLCYSTEINE

(71) Applicants: Seabelife, Roscoff (FR); Universite de Rennes, Rennes (FR); Institut National de la Santé et de la Recherche Médicale, Paris (FR); Ecole des Hautes Études en Santé Publique (EHESP), Rennes Cedex (FR)

(72) Inventors: Mélanie SIMOES EUGENIO, Rennes (FR); Marie-Thérèse DIMANCHE-BOITREL, Melesse (FR); Sophie BELAL, Plougar (FR); Morgane ROUSSELOT, Plouenan (FR)

(73) Assignees: Seabelife, Roscoff (FR); Universite de Rennes, Rennes (FR); Institut National de la Santé et de la Recherche Médicale, Paris (FR); Ecole des Hautes Études en Santé Publique (EHESP), Rennes Cedex (FR)

(21) Appl. No.: 19/132,457

(22) PCT Filed: Dec. 4, 2023
(86) PCT No.: PCT/EP2023/084156

§ 371 Date: May 23, 2025

(30) Foreign Application Priority Data
Dec. 2, 2022 (EP) 22306783.6

(51) Int. Cl. A61K 31/198; A61K 31/404; A61K 31/437; A61K 31/444; A61K 31/4545; A61K 31/496; A61K 31/506; A61K 31/5377; A61P 1/16

(52) U.S. Cl. CPC A61K 31/198; A61K 31/404; A61K 31/437; A61K 31/444; A61K 31/4545; A61K 31/496; A61K 31/506; A61K 31/5377; A61P 1/16

(57) ABSTRACT

The present invention relates to relates to a pharmaceutical composition comprising a combination of N-acetylcysteine with at least one inhibitor of regulated necrotic cell death, such as necroptosis and/or ferroptosis.

The present invention relates to a pharmaceutical composition comprising a combination of N-acetylcysteine with at least one inhibitor of regulated necrotic cell death, such as necroptosis and/or ferroptosis.

Necroptosis, a programmed cell death route, is clearly distinct from apoptosis as it does not involve key apoptosis regulators, such as caspases, Bcl-2 family members or cytochrome c release from mitochondria. “Necroptosis” is a specialized biochemical pathway of programmed necrosis that depends notably on the serine/threonine kinase activity of RIPK1 (Receptor-Interacting Protein Kinase 1). The ground-breaking finding that necroptosis is a genetically controlled process led to the hypothesis that this programmed cell-death is ‘druggable’, an emerging breakthrough that carries the potential to revolutionize every day clinical medicine [Linkermann and Green, N. Eng. J. Med. 2014, 370(5), 455-465]. Indeed, molecular targets, including RIPK1 (Receptor Interacting Protein 1), RIPK3 and MLKL (Mixed Lineage Kinase domain-Like), have convincingly been shown to contribute to multiple disorders where necroptosis is of central pathophysiological relevance.

Necroptosis plays important role in the pathogenesis of various diseases across the body, including conditions of the neurologic, cardiovascular, pulmonary, and gastrointestinal systems. Necroptosis also plays a role in infectious and autoimmune diseases. Necroptosis was also reported to mediate organ rejection in particular in cardiac and renal allografts (Khoury et al. Am j pathology, 2020; Jouan-Lanhouet et al. semin. Cell dev. Biol. 2014).

Ferroptosis is a new type of non-apoptotic regulated cell death that was first described in 2012, and usually involves high intracellular levels of free iron and lipid peroxidation. This death pathway is directly linked to the ability of the cell to regulate its internal oxidative stress, notably via the activity of the lipid repair enzyme glutathione peroxidase 4 (GPX4). The failure of the glutathione-dependent antioxidant defenses causes an accumulation of lipid-based reactive oxygen species (ROS), which result notably from lipid peroxidation by Fe²⁺, through the Fenton's reaction, leading to membrane damage and cell death.

Recent studies show that ferroptosis is involved in the pathophysiology of many human diseases [Li et al., Cell Death Dis., 2020, 11(88); Tang et al., Cell Research, 2021, 31:107-125; Sun et al., Biomed. Pharmacother., 2020, 127, 110108], affecting in particular the heart, the brain, the nervous system, the eyes, the gastrointestinal system, the liver, the skin, the kidneys, the lungs, the bowel the pancreas or the whole body. Ferroptosis involves three primary metabolisms including thiol, lipid and iron leading to an iron-dependent generation of lipid peroxidation and, ultimately to cell death.

Hallmarks of ferroptosis were used as key elements to define ferroptosis-associated disease biomarkers. Ferroptosis is an iron-dependent regulated tissue necrosis mainly caused by unrestricted lipid peroxidation and subsequent membrane damage. The modifications of the physiological levels of the following components were reported as associated with ferroptosis: iron, reactive oxygen species, ROS (including lipid ROS such as 4-Hydroxynonenal (4-HNE) and malondialdehyde (MDA), and oxidized phospatidylethanolamine (oxPE) species followed by oxidized phosphatidylserine (oxPS) and oxidized phosphatidylinositol (oxPI) [Wiernicki et al., Cell Death Dis., 2020, 11(922)]) and related peroxide detoxification molecules (including the thiol-containing compound gluthation, GSH, or Coenzyme Q10, also known as ubiquinone), and the long-chain-fatty-acid-CoA ligase 4 (ACSL4) [Chen X. et al., Front. in Cell and Dev. Biol., 2021, 9(637162)]. These key biochemical ferroptosis biomarkers can be measured and quantified by assays in bodily fluids (blood, plasma, serum, urine, cerebrospinal fluid) or highlighted by immunohistochemistry labeling on biopsies of damaged tissues.

Depending on both the pathology and the damaged organ, several among ferroptosis-associated biomarkers could vary (increase > or decrease <) in quantity and/or activity relative to normal physiological thresholds. Here we only described reference values for serum:
(1) Iron metabolism (by measuring iron and ferritin levels in serum) is over the physiological thresholds (serum iron, in male >180 μg/dl, in female >160 μg/dl; [Pagana et al., Mosby's Diagnostic and Laboratory Test Reference—Elsevier eBook on VitalSource, 14th Edition, Elsevier, 2019, ISBN: 9780323609678]), ferritin, in male >300 ng/ml, in female >200 ng/ml, [Wang et al., Biochim Biophy. Acta, 2010, 1800(8): 760-769]);
(2) Glutathione redox status (by measuring reduced glutathione (GSH) and oxidized glutathione (GSSG) as well as glutathione peroxidase activity (GPx) in plasma using ELISA) (GSH<717 μmol/L, GSSG>5.32 μmol/L; ratio GSH/GSSG<156; GPx, in male <20 UI/gHb, in female <26 UI/gHb), [Haleng J. et al., Rev. Med. Liege, 2007]);
(3) Oxidative stress (by measuring levels of total Q10 and reduced and active form of Q10 (Q10H2) in plasma (in male Q10<3.44 μmol/1 and Q10H2<3.04 μmol/l; in female Q10<1.88 μmol/l and Q10H2<1.64 μmol/1, [Kaikkonen et al., Scand J Clin Lab Invest, 1999, 59: 457-466]);
(4) Lipid peroxidation (measured by detection of 4-Hydroxynonenal (4-HNE) and malondialdehyde (MDA) adducts) is over the physiological thresholds (>10 μmol/L for 4-HNE [Chen and Niki, IUBMB Life, 2008, 58(372-373)] and >3 μmol/L for MDA using thiobarbituric acid method [Banjare et al., J. Sci. Soc., 2017; 44(137-9)]).

Note here that the upregulation of ACSL4 enzyme level in damaged organ tissues was also reported as putative biomarker of ferroptosis (ACSL4 expression can be monitored by transcriptomic and proteomic approaches).

Pathologies associated with ferroptosis affecting the heart include myocardial ischemia-reperfusion injury, notably occurring after artery ligation, cardiomyopathy, notably doxorubicin-induced cardiomyopathy [Li et al., 2020], and cardiovascular disease, notably aortic dissection [Chen et al., Pharmacol. Res., 2022, 177, 106122], among others [Li et al., Free Radic. Biol. Med., 2020, 160, 303-318; Qin et al., Biomed. Pharmacother., 2021, 141, 111872].

Pathologies associated with ferroptosis affecting the central nervous system include strokes, notably ischemic stroke [Li et al., 2020] or hemorrhagic stroke [Li et al., JCI Insight, 2017, 2(7):e90777], traumatic brain injury [Xie et al., CNS Neurosci Ther., 2019, 25:465-475], contusion spinal cord injury [Zhang et al., Neural Regen. Res., 2019, 14(3):532], epilepsy, including mitochondrial disease-related epilepsy and intractable epilepsy [Kahn-Kirby et al., PLoS One., 2019, 14(3)], and neurodegenerative disorders, in particular chronic neurodegenerative disorders, more particularly Alzheimer's disease [Li et al., 2020], Huntington's disease [Mi et al., Neuromolecular Med., 2019, 21, 110-119], Parkinson's disease [Do Van et al., Neurobiol Dis., 2016, 94: 169-78], amyotrophic lateral sclerosis (Charcot's disease) [Li et al., 2020], multiple sclerosis [Luoqian et al., Cell Mol Immunol., 2022, 19(8), 913-924], Friedreich's ataxia [Cotticelli et al., J Pharmacol Exp Ther., 2019, 369(1): 47-54], periventriculor leukomalacia [Skouta et al., J. Am. Chem. Soc., 2014, 136, 4551-4556] and dementia, which may be linked to one or several of the previous pathologies.

Pathologies associated with ferroptosis affecting the eyes include vision loss, in particular due to cataract [Wei et al., Free Radic Biol Med., 2021, 167, 94-108], and retinal disorders, notably Stargardt disease and age-related macular degeneration (AMD), in particular dry AMD [Sun et al., Invest Ophth Vis Sci., 2018, 59(9), 2482; Chen et al., J. Biol. Chem., 2021, 296, 100187].

Pathologies associated with ferroptosis affecting the liver include chronic liver diseases as well as acute liver injury and acute liver failure. Among chronic liver diseases, mention should be made of non-alcoholic steatohepatitis (NASH) [Qi et al., Am J Pathol., 2020, 190(1)], chronic infections such as hepatitis B and C [Cappelletti et al., Int J Mol Sci., 2020, 21(14)] and alcoholic cirrhosis [Zhou et al., Hepatol Commun., 2019, 3(5)]. Acute liver failure may notably result from a drug-induced liver injury (DILI), such as acetaminophen (APAP)-induced liver injury [Yamada et al., Cell Death Dis., 2020, 11(2)], from an ischemia-reperfusion injury induced by a septic or hemorrhagic shock [Friedmann Angeli et al., Nat Cell Biol., 2014, 16(12):1180-91], from fulminant viral hepatitis, from auto-immune origin or from alcohol intake.

Pathologies associated with ferroptosis affecting the skin include skin inflammatory diseases, such as psoriasis [Li et al., Cell Death Dis., 2020, 11(88)], and toxic epidermal necrolysis (Lyell syndrome) [Zhang et al., J Invest Dermatol., 2020, 140(7), S79].

Pathologies associated with ferroptosis affecting the kidneys include acute kidney injury (AKI) (also known as acute renal failure), such as crystal (oxalate)-, folic acid (FA)-induced AKI [Martin-Sanchez et al., 2017] and cisplatin-induced AKI [Deng et al., J Clin Invest., 2019, 129(11); Mishima et al., J Am Soc Nephrol., 2020, 31(2); Hu et al., Cell Death Dis., 2020, 11(1)], renal ischemia-reperfusion injury [Li et al., 2020], and acute tubular necrosis [Friedmann Angeli et al., 2014].

Pathologies associated with ferroptosis affecting the lungs include chronic obstructive pulmonary disease (COPD) [Yoshida et al., Nat Commun., 2019, 10, 3145], bronchial asthma [Tao et al., Oxid Med Cell Longev., 2020], lung injury caused by a bacterial infection, notably by Pseudomonas aeruginosa [Dar et al., J Clin Invest., 2018, 128(10), 4639-4653] or Mycobacterium tuberculosis [Amaral et al., J Exp Med., 2019, 216(3): 556-570] and pulmonary fibrosis, such as radiation induced-lung fibrosis (RILF) [Li et al., J Inflamm., 2019, 16:11] and paraquat-induced pulmonary damage [Rashidipour et al., Toxicology, 2020, 433-434:152407].

Pathologies associated with ferroptosis affecting the gut include necrotizing enterocolitis [Subramanian et al., Acta Physiologica Sinica, 2020, 72(3)] and inflammatory bowel diseases, such as Crohn's disease [Mayr et al., Nat Commun., 2020, 11(1)] and ulcerative colitis.

Pathologies associated with ferroptosis affecting the whole body include haemochromatosis [Imoto et al., Transfus Apher Sci., 2018, 57(4), 524-531], β-thalassemia [Sposi, N. M., Oxidative Stress and Iron Overload in β-Thalassemia: An Overview, 2019, DOI: 10.5772/intechopen. 90492], hemolytic disorders [Youssef et al., 2019, Ferroptosis in Hemolytic Disorders. In: Tang D. (eds) Ferroptosis in Health and Disease. Springer, Cham.], cytokinic storm during a viral infection [Edeas et al., Int J Infect Dis., 2020, 97; Yang and Lai, Cell Death Discov., 2020, 6], radiation-induced necrosis [Wu et al., Front Oncol., 2020, 10], rheumatoid arthritis [Xie et al., Inflammation., 2020, doi: 10.1007/s10753-020-01338-2], type I diabetes [Bruni et al., Cell Transplant., 2018, 27(6)], insulin resistance related to obesity, and pathologies related to stress-induced premature tissue senescence, such as atherosclerosis [Bai et al., Free Radic Biol Med., 2020, 160], hypertension [Yang et al., Clin Exp Hypertens., 2020, 42(8)] and type II diabetes [Li et al., Nutrients., 2020, 12(10)].

Therefore, inhibition of regulated necrotic cell death is a new and attractive therapeutic strategy for disorders associated with it.

The inventors have previously disclosed two new classes of regulated necrosis inhibitors (also designated herein as “inhibitors of regulated necrotic cell death”), the first one consisting of sibiriline derivatives (WO 2017/064217, WO 2017/064216, and WO 2022/157392), and the second one consisting of nigratine derivatives (WO 2018/073321).

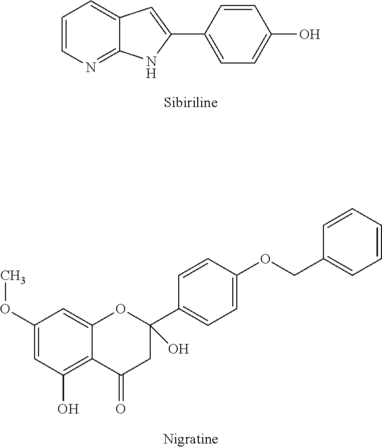 [SMILES: Oc1ccc(-c2cc3cccnc3[nH]2)cc1 ; COc1cc(O)c2c(c1)OC(O)(c1ccc(OCc3ccccc3)cc1)CC2=O]

Now, the inventors have discovered that, surprisingly, when such regulated necrosis inhibitors (inhibitors of regulated necrotic cell death) are combined with N-acetylcysteine (NAC) or a derivative thereof, the combination produces cell protection effects that indicate super-additivity (i.e. synergy), achieving a much better therapeutic efficacy than when each active ingredient is used alone.

Hence, the present invention relates to a pharmaceutical composition comprising a combination of N-acetylcysteine (NAC) and/or a pharmaceutical salt and/or a derivative thereof with at least one inhibitor of regulated necrotic cell death, such as necroptosis and/or ferroptosis.

N-acetylcysteine (also named acetylcysteine or N-acetyl-L-cysteine, CAS number 616-91-1) corresponds to the following chemical formula:

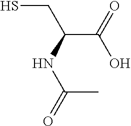

N-acetylcysteine The main therapeutic applications of NAC based on its pharmacological activity are listed below:
In pneumology, NAC is a mucolytic-type mucomodifier. It acts on the gel phase of mucus, presumably by breaking the disulfide bridges of glycoproteins, and thus promotes expectoration.
In ophthalmology, NAC is an inhibitor of collagenase, a proteolytic enzyme secreted in large quantities during any damage to the epithelium and causing the degradation of polypeptide fibers of corneal collagen.
In toxicology, acetylcysteine is a precursor of glutathione, which can enter cells. This is the main way in which it protects hepatocytes. In fact, glutathione neutralizes the electrophilic entities produced by the metabolism of paracetamol.
In neurology, several initial studies showed that administration of NAC increased glutathione levels in the brain. The benefit of NAC has recently been confirmed as part of a standard treatment for patients with Parkinson's disease. The study found improved levels of dopamine, the main neurotransmitter specifically reduced in the disease, as well as improved mental and physical abilities in patients [Monti et al., Clin Pharmacol Ther., 2019, 106(4), 884-890].

Pharmaceutically acceptable salts of NAC or derivative thereof such as N-acetylcysteine amide, N-acetylcysteine ethyl ester, N-acetylcysteine methyl ester include, but are not limited to, those formed with free amino groups such as those derived from hydrochloric, phosphoric, sulfuric, acetic, oxalic, tartaric acids, and the like, and those formed with free carboxyl groups such as those derived from sodium, potassium, ammonium, calcium, ferric hydroxides, isopropylamine, triethylamine, 2-(ethylamino)ethanol, histidine, procaine and the like.

In the framework of the present invention, the term “derivative of NAC” is intended to mean any compound which is structurally related to NAC and which possesses a similar pharmacological activity. For instance, the following compound, known as N-acetylcysteine amide (also named NAC amide or N-acetyl-L-cysteine amide or NACA, CAS number 38520-57-9) [Sunitha et al., Clin Pharmacol Ther., 2013, 47(5), 884-890] is a derivative of NAC:

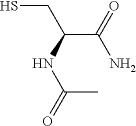

N-acetylcysteine amide In particular, the derivative of NAC of a pharmaceutical composition according to the invention is N-acetylcysteine amide.

For instance, the following compound, known as N-acetylcysteine ethyl ester (also named NAC ethyl ester or N-acetyl-L-cysteine ethyl ester, CAS number 59587-09-6) is a derivative of NAC:

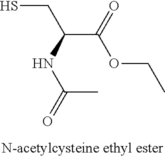

In particular, the derivative of NAC of a pharmaceutical composition according to the invention is N-acetylcysteine ethyl ester.

For instance, the following compound, known as N-acetylcysteine methyl ester (also named NAC methyl ester N-acetyl-L-cysteine methyl ester, CAS number 118398-49-5) is a derivative of NAC:

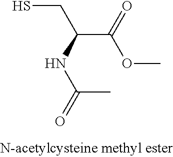

In particular, the derivative of NAC of a pharmaceutical composition according to the invention is N-acetylcysteine methyl ester.

Particularly, the invention relates to a pharmaceutical composition comprising a combination of N-acetylcysteine (NAC) and/or N-acetylcysteine amide and/or N-acetylcysteine ethyl ester and/or N-acetylcysteine methyl ester and/or a pharmaceutical salt thereof with at least one inhibitor of regulated necrotic cell death, such as necroptosis and/or ferroptosis.

For the purpose of the invention, the term “inhibitor of regulated necrotic cell death” refers to a compound which allows to preserve at least partially cells from a regulated necrotic death. In particular, when cells are exposed to a regulated necrotic cell death inducer, treatment with the inhibitor allows to improve cell viability, which can be easily verified by the skilled person using methods well known in the art, such as those described in the examples of the present description.

The inhibitor of regulated necrotic cell death is advantageously a ferroptosis and/or a necroptosis inhibitor. In a particular embodiment, the inhibitor of regulated necrotic cell death is a ferroptosis inhibitor, preferably a ferroptosis and necroptosis inhibitor.

Tests for identifying ferroptosis inhibitors are for instance disclosed in WO2022157392, from pages 69 to 81 (experimental part, section “II. Biological Activity of the compounds of the invention). A preferred model is SH-SY5Y human neuroblastoma cell line treated with ferroptosis inducer RSL3 or erastin. A ferroptosis inhibitor is characterized by its ability to protect cells from cell death in a dose-dependent manner and to decrease lipid peroxidation induced by a ferroptosis inducer such as RSL3 or erastin for example in the model SH-SY5Y. Tests for identifying necroptosis inhibitors are for instance disclosed in page 29 of EP3362450B1, specially in paragraph [178]. A necroptosis inhibitor is characterized by its ability to protect cells from cell death induced by a necroptosis inducer, in a dose-dependent manner in this test, for example with an EC₅₀ of 25 μM or less (EC₅₀ is the half maximal effective concentration of the drug).
Examples of known ferroptosis and/or a necroptosis inhibitor are Resveratrol (CAS number 501-36-0), Ferrostatin 1 (CAS Number: 347174-05-4) or Liproxstatin (CAS Number 950455-15-9), and NECIF (described in particular in (Tonnus et al. Nat Commun 12, 4402 (2021), DOI: 10.1038/s41467-021-24712-6). In particular, the inhibitor of regulated necrotic cell death inhibitor is NEC1F.

The inhibitor of regulated necrotic cell death (hereinafter, “the inhibitor”) of a pharmaceutical composition according to the invention may be, in particular, sibiriline, nigratine or a derivative thereof.

### Sibiriline and Derivatives Thereof

In a particular embodiment, the inhibitor of a pharmaceutical composition according to the invention is sibiriline or a derivative thereof. Said inhibitor may thus correspond to any compound disclosed in WO 2017/064217, WO 2017/064216 or WO 2022/157392.

In this particular embodiment, the inhibitor is preferably a compound of general formula (I)

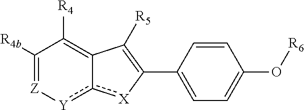

or a pharmaceutically acceptable salt and/or solvate thereof,
wherein:

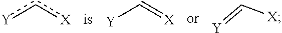

(i) when

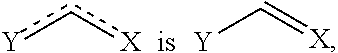

X is N, Y is N(R₂) and Z is C(H);
(ii) when

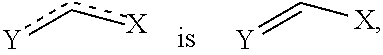

X is N(R₁), and
Y is N or N⁺(O⁻) and Z is C(R₃), or
Y is CH and Z is N, or
Y and Z are CH;
   and wherein:
R₁ and R₂ represent, independently of each other, a hydrogen atom, CN, NO₂, OR₇, SR₈, NR₉R₁₀, C(O)R₁₁, CO₂R₁₂, OC(O)R₁₃, NR₁₄C(O)R₁₅, C(O)NR₁₆R₁₇, S(O)Rₛ, SO₂Rₛ′, a (C₁-C₆)alkyl, a (C₁-C₆)haloalkyl, a —(C₁-C₆)alkyl-[O—(C₁-C₆)alkyl]ₘ-NR_{N1}R_{N2} group with m ranging from 1 to 6, an aryl, an aryl-(C₁-C₆)alkyl, a heterocyclyl, or a heterocyclyl-(C₁-C₆)alkyl group, wherein said aryl or heterocyclyl group is optionally substituted by one or more substituents selected from the group consisting of a halogen atom, CN, NO₂, OR₁₈, SR₁₉, NR₂₀R₂₁, C(O)R₂₂, CO₂R₂₃, OC(O)R₂₄, NR₂₅C(O)R₂₆, C(O)NR₂₇R₂₈, a (C₁-C₆)alkyl and a (C₁-C₆)haloalkyl group;
R₃, R₄, R_{4b} and R₅ represent, independently of each other, a hydrogen atom, a halogen atom, CN, OR₂₉, SR₃₀, NR₃₁R₃₂, C(O)R₃₃, CO₂R₃₄, OC(O)R₃₅, NR₃₆C(O)R₃₇, C(O)NR₃₈R₃₉, a (C₁-C₆)alkyl, a (C₁-C₆)haloalkyl group, said alkyl or haloalkyl group being optionally substituted by one or more substituents selected from the group consisting of OR₄₀, SR₄₁ and NR₄₂R₄₃, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, CN, NO₂, OR₄₄, SR₄₅, NR₄₆R₄₇, C(O)R₄₈, CO₂R₄₉, OC(O)R₅₀, NR₅₁C(O)R₅₂, C(O)NR₅₃R₅₄, a (C₁-C₆)alkyl and a (C₁-C₆)haloalkyl group;
R₆ represents a hydrogen atom, a (C₁-C₆)alkyl, an aryl-(C₁-C₆)alkyl, a heterocyclyl-(C₁-C₆)alkyl, a —(C₁-C₆)alkyl-[O—(C₁-C₆)alkyl]ₘ—NR_{N′1}R_{N′2} group with m′ ranging from 1 to 6, or a (C₁-C₆)alkylcarbonyl group, said (C₁-C₆)alkyl, aryl-(C₁-C₆)alkyl, and (C₁-C₆)alkylcarbonyl group being optionally substituted with one or more substituents selected from the group consisting of OH, SH, NH₂, a (C₁-C₆)alkoxy, a (C₁-C₆)thioalkoxy, a (C₁-C₆)alkylamino and a di((C₁-C₆)alkyl)amino group;
Rₛ and Rₛ′ represent, independently of each other a (C₁-C₆)alkyl, an aryl or an aryl-(C₁-C₆)alkyl group;
R₇-R₁₀, R₁₂, R₁₄ and R₁₆-R₁₇ represent, independently of each other, a hydrogen atom, a (C₁-C₆)alkyl, an aryl or an aryl-(C₁-C₆)alkyl group;
R₁₁, R₁₃ and R₁₅ represent, independently of each other, a hydrogen atom, a (C₁-C₆)alkyl, an aryl, an aryl-(C₁-C₆)alkyl, a (C₁-C₆)alkoxy, a (C₁-C₆)alkylamino or a di((C₁-C₆)alkyl)amino group;
R₁₈ to R₂₈ represent, independently of each other, a hydrogen atom, a (C₁-C₆)alkyl or an aryl group;
R₂₉ to R₃₉ represent, independently of each other, a hydrogen atom, a (C₁-C₆)alkyl, heterocyclyl-(C₁-C₆)alkyl, an aryl or an aryl-(C₁-C₆)alkyl group, said aryl group being optionally substituted by one or more substituents selected from the group consisting of a halogen atom, CN, NO₂, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group;
R₄₀ to R₄₃ represent, independently of each other, a hydrogen atom or a (C₁-C₆)alkyl group;
R₄₄ to R₅₄ represent, independently of each other, a hydrogen atom, a (C₁-C₆)alkyl or an aryl group;
R₅₅ to R₆₅ represent, independently of each other, a hydrogen atom, a (C₁-C₆)alkyl, aryl-(C₁-C₆)alkyl group or an aryl group; and
   R_{N1}, R_{N′1}, R_{N2} and R_{N′2} represent, independently of each other, a hydrogen atom, a (C₁-C₆)alkyl group, an aryl-(C₁-C₆)alkyl group or an aryl group.
Preferably, in the compound of the following general formula (I) wherein: R₁ and R₂ represent, independently of each other, a hydrogen atom, CN, NO₂, OR₇, SR₈, NR₉R₁₀, C(O)R₁₁, CO₂R₁₂, OC(O)R₁₃, NR₁₄C(O)R₁₅, C(O)NR₁₆R₁₇, S(O)Rₛ, SO₂Rₛ′, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, CN, NO₂, ORIS, SR₁₉, NR₂₀R₂₁, C(O)R₂₂, CO₂R₂₃, OC(O)R₂₄, NR₂₅C(O)R₂₆, C(O)NR₂₇R₂₈, a (C₁-C₆)alkyl and a (C₁-C₆)haloalkyl group;
R₃, R₄, R_{4b} and R₅ represent, independently of each other, a hydrogen atom, a halogen atom, CN, OR₂₉, SR₃₀, NR₃₁R₃₂, C(O)R₃₃, CO₂R₃₄, OC(O)R₃₅, NR₃₆C(O)R₃₇, C(O)NR₃₈R₃₉, a (C₁-C₆)alkyl, a (C₁-C₆)haloalkyl group, said alkyl or haloalkyl group being optionally substituted by one or more substituents selected from the group consisting of OR₄₀, SR₄₁ and NR₄₂R₄₃, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, CN, NO₂, OR₄₄, SR₄₅, NR₄₆R₄₇, C(O)R₄₈, CO₂R₄₉, OC(O)R₅₀, NR₅₁C(O)R₅₂, C(O)NR₅₃R₅₄, a (C₁-C₆)alkyl and a (C₁-C₆)haloalkyl group;
R₆ represents a hydrogen atom, a (C₁-C₆)alkyl, an aryl-(C₁-C₆)alkyl or a —CH₂—CH₂—O—CH₂—CH₂—NH₂ group, or a (C₁-C₆)alkylcarbonyl group optionally substituted with one or more substituents selected from the group consisting of OH, SH, NH₂, a (C₁-C₆)alkoxy, a (C₁-C₆)thioalkoxy, a (C₁-C₆)alkylamino and a di((C₁-C₆)alkyl)amino group;
Rₛ and Rₛ′ represent, independently of each other a (C₁-C₆)alkyl, an aryl or an aryl-(C₁-C₆)alkyl group;
R₇-R₁₀, R₁₂, R₁₄ and R₁₆-R₁₇ represent, independently of each other, a hydrogen atom, a (C₁-C₆)alkyl, an aryl or an aryl-(C₁-C₆)alkyl group;
R₁₁, R₁₃ and R₁₅ represent, independently of each other, a hydrogen atom, a (C₁-C₆)alkyl, an aryl, an aryl-(C₁-C₆)alkyl, a (C₁-C₆)alkoxy, a (C₁-C₆)alkylamino or a di((C₁-C₆)alkyl)amino group;
R₁₈ to R₂₈ represent, independently of each other, a hydrogen atom, a (C₁-C₆)alkyl or an aryl group;
R₂₉ to R₃₉ represent, independently of each other, a hydrogen atom, a (C₁-C₆)alkyl, an aryl or an aryl-(C₁-C₆)alkyl group, said aryl group being optionally substituted by one or more substituents selected from the group consisting of a halogen atom, CN, NO₂, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group;
R₄₀ to R₄₃ represent, independently of each other, a hydrogen atom or a (C₁-C₆)alkyl group;
R₄₄ to R₅₄ represent, independently of each other, a hydrogen atom, a (C₁-C₆)alkyl or an aryl group; and
R₅₅ to R₆₅ represent, independently of each other, a hydrogen atom, a (C₁-C₆)alkyl or an aryl group.

For the purpose of the invention, the term “pharmaceutically acceptable” is intended to mean what is useful to the preparation of a pharmaceutical composition, and what is generally safe and non-toxic, for a pharmaceutical use.

The term “pharmaceutically acceptable salt or solvate” is intended to mean, in the framework of the present invention, a salt or solvate of a compound which is pharmaceutically acceptable, as defined above, and which possesses the pharmacological activity of the corresponding compound.

The pharmaceutically acceptable salts comprise:
(1) acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and
(2) base addition salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

Acceptable solvates include conventional solvates such as those formed during the last step of the preparation of the compounds due to the presence of solvents.

The term “halogen”, as used in the present invention, refers to a fluorine, bromine, chlorine or iodine atom.

The terms “(C₁-C₆)alkyl”, as used in the present invention, refers to a straight or branched saturated hydrocarbon chain containing from 1 to 6 carbon atoms including, but not limited to, methyl, ethyl, n-propyl, iso-propyl, n-butyl, iso-butyl, sec-butyl, t-butyl, n-pentyl, n-hexyl, and the like.

The term “(C₁-C₆)haloalkyl”, as used in the present invention, refers to a (C₁-C₆)alkyl group as defined above in which part or all of the hydrogen atoms is replaced with a halogen atom as defined above. This means that the (C₁-C₆)alkyl group is substituted by at least one halogen atom. It can be for example a trifluoromethyl group.

The term “aryl”, as used in the present invention, refers to an aromatic hydrocarbon group comprising preferably 6 to 10 carbon atoms and comprising one or more, notably 1 or 2, fused rings, such as, for example, a phenyl or naphtyl group, advantageously a phenyl group.

The term “heterocyclic” as used in the present invention refers to a saturated, unsaturated (i.e. not aromatic) or aromatic monocyclic or bicyclic group comprising two fused, bridged or spiro rings, preferably fused rings, advantageously comprising 5 to 10, notably 5 or 6, atoms in each ring, in which the atoms of the ring(s) comprise one or more, advantageously 1 to 3, heteroatoms selected from O, S and N, preferably O and N, the remainder being carbon atoms.

A saturated heterocyclic group is more particularly a 5- or 6-membered saturated monocyclic heterocyclic group such as a pyrrolidinyl, tetrahydrofuranyl, tetrahydrothiophenyl, thiazolidinyl, isothiazolidinyl, oxazolidinyl, isoxazolidinyl, imidazolidinyl, pyrazolidinyl, triazolidinyl, piperidinyl, piperazinyl, morpholinyl or thiomorpholinyl group.

An unsaturated heterocyclic group is more particularly an unsaturated monocyclic or bicyclic heterocyclic group, each cycle comprising 5 or 6 members, such as a pyrrolinyl, dihydrofuranyl, dihydrothiophenyl, thiazolinyl, isothiazolinyl, oxazolinyl, isoxazolinyl, imidazolinyl, pyrazolinyl, triazolinyl, dihydropyridinyl, tetrahydropyridinyl, dihydropyrimidinyl, tetrahydropyrimidinyl, dihydropyridazinyl, tetrahydropyridazinyl, dihydropyrazinyl, tetrahydropyrazinyl, dihydrotriazinyl, tetrahydrotriazinyl, indolinyl, 2,3-dihydrobenzofuranyl, 2,3-dihydrobenzothiophenyl, 1,3-benzodioxolyl, 1,3-benzoxathiolyl, benzoxazolinyl, benzothiazolinyl, benzimidazolinyl, chromanyl or chromenyl group.

An aromatic heterocyclic group, also called heteroaryl group, is more particularly an aromatic monocyclic or bicyclic heterocyclic group, each cycle comprising 5 or 6 members, such as a pyrrolyl, furanyl, thiophenyl, thiazolyl, isothiazolyl, oxazolyl, isoxazolyl, imidazolyl, pyrazolyl, triazolyl, pyridinyl, pyrimidinyl, pyridazinyl, pyrazinyl, triazinyl (such as 1,3,5-triazinyl), indolyl, benzofuranyl, benzothiophenyl, benzothiazolyl, benzoxazolyl, benzimidazolyl, indazolyl, benzotriazolyl, purinyl, quinolinyl, isoquinolinyl, cinnolinyl, quinazolinyl or quinoxalinyl group.

The term “aryl-(C₁-C₆)alkyl”, as used in the present invention, refers to an aryl group as defined above bound to the molecule via a (C₁-C₆)alkyl group as defined above. In particular, the —(C₁-C₆)alkyl-aryl group is a benzyl group.

The term “heterocyclyl —(C₁-C₆)alkyl”, as used in the present invention, refers to a heterocyclyl group as defined above bound to the molecule via a (C₁-C₆)alkyl group as defined above. In particular, the —(C₁-C₆)alkyl-heterocyclyl group is 5- or 6-membered saturated monocyclic heterocyclic group as defined above bound to the molecule via a (C₁-C₆)alkyl group as defined above.

The term “(C₁-C₆)alkylcarbonyl”, as used in the present invention, refers to a (C₁-C₆)alkyl group as defined above bound to the molecule via a —C(═O)— group, including, but not limited to, acetyl, propionyl, butanoyl, pentanoyl, hexanoyl and the like.

The term “(C₁-C₆)alkoxy”, as used in the present invention, refers to a (C₁-C₆)alkyl group as defined above bound to the molecule via an oxygen atom, including, but not limited to, methoxy, ethoxy, n-propoxy, iso-propoxy, n-butoxy, iso-butoxy, sec-butoxy, t-butoxy, n-pentoxy, n-hexoxy, and the like.

The term “(C₁-C₆)thioalkoxy”, as used in the present invention, refers to a (C₁-C₆)alkyl group as defined above bound to the molecule via a sulfur atom, including, but not limited to, thiomethoxy, thioethoxy, n-thiopropoxy, iso-thiopropoxy, n-thiobutoxy, iso-thiobutoxy, sec-thiobutoxy, t-thiobutoxy, n-thiopentoxy, n-thiohexoxy, and the like.

The term “(C₁-C₆)alkylamino”, as used in the present invention, refers to a —NHAlk group with Alk representing a (C₁-C₆)alkyl group as defined above, including, but not limited to, methylamino, ethylamino, n-propylamino, iso-propylamino, n-butylamino, iso-butylamino, sec-butylamino, t-butylamino, n-pentylamino, n-hexylamino, and the like.

The term “di(C₁-C₆)alkylamino”, as used in the present invention, refers to a —NAlk₁Alk₂ group with Alk₁ and Alk₂ representing, independently of one another, a (C₁-C₆)alkyl group as defined above, including, but not limited to, dimethylamino, diethylamino, ethylmethylamino and the like.

According to a particular embodiment of the present invention, R₁ represents a hydrogen atom, CN, NO₂, OR₇, SR₈, NR₉R₁₀, C(O)R₁₁, CO₂R₁, OC(O)R₁₃, NR₁₄C(O)R₁₅, C(O)NR₁₆R₁₇, S(O)Rₛ, SO₂Rₛ′, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group (which may be part of a aryl-(C₁-C₆)alkyl or heterocyclyl-(C₁-C₆)alkyl group) is optionally substituted by one or more substituents, notably one susbtituent, selected from the group consisting of a halogen atom, CN, NO₂, OR₁₈, SR₁₉, NR₂₀R₂₁, a (C₁-C₆)alkyl and a (C₁-C₆)haloalkyl, notably a halogen atom, NO₂, OR₁₈, a (C₁-C₆)alkyl and a (C₁-C₆)haloalkyl group, in particular NO₂ and OR₁₈, and wherein R₁₈ to R₂₁ represent, independently of each other, a hydrogen atom or a (C₁-C₆)alkyl group.

According to another particular embodiment of the present invention, R₁ represents a hydrogen atom, CN, NO₂, OR₇, SR₈, NR₉R₁₀, C(O)R₁₁, CO₂R₁₂, OC(O)R₁₃, NR₁₄C(O)R₁₅, C(O)NR₁₆R₁₇, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group is optionally substituted by one or more substituents as defined above.

According to still another particular embodiment of the present invention, R₁ represents a hydrogen atom, CN, OR₇, C(O)R₁₁, CO₂R₁₂, OC(O)R₁₃, SO₂Rₛ′, a (C₁-C₆)alkyl, a heterocyclyl or a heterocyclyl-(C₁-C₆)alkyl group, wherein said heterocyclyl group (which may be part of a heterocyclyl-(C₁-C₆)alkyl group) is optionally substituted by one or more substituents, notably one substituent, selected from the group consisting of a halogen atom, CN, NO₂, OR₁₈, SR₁₉, NR₂₀R₂₁, a (C₁-C₆)alkyl and a (C₁-C₆)haloalkyl group, notably a halogen atom, NO₂, OR₁₈, a (C₁-C₆)alkyl and a (C₁-C₆)haloalkyl group, in particular NO₂ and OR₁₈, preferably said heterocyclyl group is optionally substituted by NO₂, and wherein R₁₈ to R₂₁ represent, independently of each other, a hydrogen atom or a (C₁-C₆)alkyl group.

According to yet another particular embodiment of the present invention, R₁ represents a hydrogen atom, CN, OR₇, C(O)R₁₁, CO₂R₁₂, OC(O)R₁₃, a heterocyclyl or a heterocyclyl-(C₁-C₆)alkyl group, wherein said heterocyclyl group is optionally substituted by one or more substituents as defined above.

In a preferred embodiment, R₁ represents a hydrogen atom or a (C₁-C₆)alkyl group, in particular a hydrogen atom or a (C₁-C₃)alkyl group, preferably a hydrogen atom.

In the above embodiments, the (C₁-C₆)alkyl group, which may be part of an aryl-(C₁-C₆)alkyl group or a heterocyclyl-(C₁-C₆)alkyl group, is preferably a (C₁-C₃)alkyl group.

In the above embodiments, the aryl group, which may be part of an aryl-(C₁-C₆)alkyl group, is preferably a phenyl group.

In the above embodiments, the heterocyclyl group, which may be part of a heterocyclyl-(C₁-C₆)alkyl group, is in particular a 5- or 6-membered, saturated, unsaturated (i.e. not aromatic) or aromatic, notably saturated or aromatic, monocyclic group, in which the atoms of the ring comprise one or more, advantageously 1 to 3, heteroatoms selected from O, S and N, preferably O and N, the remainder being carbon atoms, such as a morpholinyl, a pyridinyl or a piperazinyl, for instance a morpholinyl or pyridinyl group.

In the above embodiments, Rₛ and Rₛ′ represent, independently of each other a (C₁-C₆)alkyl, an aryl or an aryl-(C₁-C₆)alkyl group, notably a (C₁-C₆)alkyl or an aryl group, in particular, an aryl group, such as a phenyl group.

In the above embodiments, R₇-R₁₀, R₁₂, R₁₄ and R₁₆-R₁₇ represent, independently of each other, a hydrogen atom, a (C₁-C₆)alkyl, an aryl or an aryl-(C₁-C₆)alkyl group, and R₁₁, R₁₃ and R₁₅ represent, independently of each other, a hydrogen atom, a (C₁-C₆)alkyl, an aryl, an aryl-(C₁-C₆)alkyl, a (C₁-C₆)alkoxy, a (C₁-C₆)alkylamino or a di((C₁-C₆)alkyl)amino group, notably a hydrogen atom, a (C₁-C₆)alkyl, an aryl, a (C₁-C₆)alkylamino or a di((C₁-C₆)alkyl)amino group, in particular a (C₁-C₃)alkyl, an aryl such as a phenyl, a (C₁-C₃)alkylamino or a di((C₁-C₃)alkyl)amino group.

In particular, in the above embodiments, R₇ to R₁₇ represent, independently of each other, a hydrogen atom, a (C₁-C₆)alkyl, an aryl or an aryl-(C₁-C₆)alkyl group, notably a hydrogen atom, a (C₁-C₆)alkyl or an aryl group, typically a hydrogen atom, a (C₁-C₃)alkyl or an aryl group, wherein the aryl group, which may be part of an aryl-(C₁-C₆)alkyl group, is preferably a phenyl group.

According to a particular embodiment of the present invention, R₂ represents a hydrogen atom, CN, NO₂, OR₇, SR₈, NR₉R₁₀, C(O)R₁₁, CO₂R₁₂, OC(O)R₁₃, NR₁₄C(O)R₁₅, C(O)NR₁₆R₁₇, S(O)Rₛ, SO₂Rₛ′, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group (which may be part of a aryl-(C₁-C₆)alkyl or heterocyclyl-(C₁-C₆)alkyl group) is optionally substituted by one or more substituents, notably by one substituent, selected from the group consisting of a halogen atom, CN, NO₂, OR₁₈, SR₁₉, NR₂₀R₂₁, a (C₁-C₆)alkyl and a (C₁-C₆)haloalkyl, notably a halogen atom, NO₂, OR₁₈, a (C₁-C₆)alkyl and a (C₁-C₆)haloalkyl group, in particular NO₂ and OR₁₈, and wherein R₁₈ to R₂₁ represent, independently of each other, a hydrogen atom or a (C₁-C₆)alkyl group.

In the above embodiment, Rₛ and Rₛ′ represent, independently of each other a (C₁-C₆)alkyl, an aryl or an aryl-(C₁-C₆)alkyl group, notably a (C₁-C₆)alkyl or an aryl group, in particular, an aryl group, such as a phenyl group.

According to another particular embodiment of the present invention, R₂ represents a hydrogen atom, CN, NO₂, OR₇, SR₈, NR₉R₁₀, C(O)R₁₁, CO₂R₁₂, OC(O)R₁₃, NR₁₄C(O)R₁₅, C(O)NR₁₆R₁₇, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group is optionally substituted by one or more substituents as defined above.

According to still another particular embodiment of the present invention, R₂ represents C(O)R₁₁, CO₂R₁₂, C(O)NR₁₆R₁₇, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group is optionally substituted by one or more substituents, notably one substituent, selected from the group consisting of a halogen atom OR₁₈, SR₁₉, NR₂₀R₂₁, a (C₁-C₆)alkyl and a (C₁-C₆)haloalkyl group, and wherein R₁₈ to R₂₁ represent, independently of each other, a hydrogen atom or a (C₁-C₆)alkyl group.

According to yet another particular embodiment of the present invention, R₂ represents CO₂R₁₂, C(O)NR₁₆R₁₇, a (C₁-C₆)alkyl, an aryl or an aryl-(C₁-C₆)alkyl group, notably CO₂R₁₂, C(O)NR₁₆R₁₇ or an aryl-(C₁-C₆)alkyl group, wherein said aryl group is optionally substituted by one or more substituents, notably one substituent, selected from the group consisting of a halogen atom, OR₁₈, SR₁₉ and NR₂₀R₂₁, notably OR₁₈, and wherein R₁₈ to R₂₁ represent, independently of each other, a hydrogen atom or a (C₁-C₆)alkyl group.

In the above embodiments, the (C₁-C₆)alkyl group, which may be part of an aryl-(C₁-C₆)alkyl group or a heterocyclyl-(C₁-C₆)alkyl group, is preferably a (C₁-C₃)alkyl group.

In the above embodiments, the aryl group, which may be part of an aryl-(C₁-C₆)alkyl group, is preferably a phenyl group.

In the above embodiments, the heterocyclyl group, which may be part of a heterocyclyl-(C₁-C₆)alkyl group, is in particular a 5- or 6-membered, saturated, unsaturated (i.e. not aromatic) or aromatic, notably saturated, monocyclic group, in which the atoms of the ring comprise one or more, advantageously 1 to 3, heteroatoms selected from O, S and N, preferably O and N, the remainder being carbon atoms, such as a morpholinyl, a pyridinyl or a piperazinyl group, notably a piperazinyl group.

In the above embodiments, R₇-R₁₀, R₁₂, R₁₄ and R₁₆-R₁₇ represent, independently of each other, a hydrogen atom, a (C₁-C₆)alkyl, an aryl or an aryl-(C₁-C₆)alkyl group, and R₁₁, R₁₃ and R₁₅ represent, independently of each other, a hydrogen atom, a (C₁-C₆)alkyl, an aryl, an aryl-(C₁-C₆)alkyl, a (C₁-C₆)alkoxy, a (C₁-C₆)alkylamino or a di((C₁-C₆)alkyl)amino group, notably a hydrogen atom, a (C₁-C₆)alkyl, an aryl, a (C₁-C₆)alkylamino or a di((C₁-C₆)alkyl)amino group, in particular a (C₁-C₃)alkyl, an aryl such as a phenyl, a (C₁-C₃)alkylamino or a di((C₁-C₃)alkyl)amino group.

In particular, in the above embodiments, R₇ to R₁₇ represent, independently of each other, a hydrogen atom, a (C₁-C₆)alkyl, an aryl or an aryl-(C₁-C₆)alkyl group, notably a hydrogen atom, a (C₁-C₆)alkyl or an aryl-(C₁-C₆)alkyl group, typically a hydrogen atom, a (C₁-C₃)alkyl or an aryl group, wherein the aryl group, which may be part of an aryl-(C₁-C₆)alkyl group, is preferably a phenyl group.

According to another particular embodiment of the present invention, R₃ represents a hydrogen atom, a halogen atom, a (C₁-C₆)alkyl group, CN, OR₂₈, SR₃₀, NR₃₁R₃₂, C(O)R₃₃, CO₂R₃₄, OC(O)R₃₅, NR₃₆C(O)R₃₇, C(O)NR₃₈R₃₉, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, OR₄₄, SR₄₅, NR₄₆R₄₇, a (C₁-C₆)alkyl and a (C₁-C₆)haloalkyl group, and wherein R₂₉ to R₃₉ represent, independently of each other, a hydrogen atom, a (C₁-C₆)alkyl, an aryl, or an aryl-(C₁-C₆)alkyl group, notably a hydrogen atom or a (C₁-C₆)alkyl group, typically a hydrogen atom or a (C₁-C₃)alkyl group.

According to another particular embodiment of the present invention, R₃ represents a hydrogen atom, a halogen atom, a (C₁-C₆)alkyl group, CN, OR₂₉, SR₃₀, NR₃₁R₃₂, C(O)R₃₃, CO₂R₃₄, OC(O)R₃₅, NR₃₆C(O)R₃₇ or C(O)NR₃₈R₃₉, wherein R₂₉ to R₃₉ represent, independently of each other, a hydrogen atom, a (C₁-C₆)alkyl, an aryl, or an aryl-(C₁-C₆)alkyl group, notably a hydrogen atom or a (C₁-C₆)alkyl group.

According to still another particular embodiment of the present invention, R₃ represents a hydrogen atom, a halogen atom, a (C₁-C₆)alkyl group, CN, OR₂₉, SR₃₀, NR₃₁R₃₂, OC(O)R₃₅, NR₃₆C(O)R₃₇, a heterocyclyl or a heterocyclyl-(C₁-C₆)alkyl group, wherein said heterocyclyl group is optionally substituted by one or more substituents selected from the group consisting of a halogen atom, OR₄₄, SR₄₅, NR₄₆R₄₇ and a (C₁-C₆)alkyl a group, preferably, R₃ represents a hydrogen atom, a halogen atom, CN, NR₃₁R₃₂, OC(O)R₃₅, a heterocyclyl or a heterocyclyl-(C₁-C₆)alkyl group, wherein said heterocyclyl group is optionally substituted by one or more substituents selected from the group consisting of a OR₄₄, SR₄₅ and NR₄₆R₄₇, notably OR₄₄, and wherein R₂₉ to R₃₇ represent, independently of each other, a hydrogen atom, a (C₁-C₆)alkyl, an aryl, or an aryl-(C₁-C₆)alkyl group, notably a hydrogen atom or a (C₁-C₆)alkyl group, typically a hydrogen atom or a (C₁-C₃)alkyl group.

In the above embodiments, R₄₄ to R₄₇ represent, independently of each other, a hydrogen atom or a (C₁-C₆)alkyl group, notably a hydrogen atom or a (C₁-C₃)alkyl group.

According to yet another particular embodiment of the present invention, R₃ represents a hydrogen atom, a halogen atom, a (C₁-C₆)alkyl group, OR₂₉, SR₃₀, NR₃₁R₃₂, C(O)R₃₃, CO₂R₃₄, OC(O)R₃₅, NR₃₆C(O)R₃₇ or C(O)NR₃₈R₃₉ preferably a hydrogen atom, a halogen atom, OR₂₉, SR₃₀, NR₃₁R₃₂, OC(O)R₃₅ or NR₃₆C(O)R₃₇, more preferably a hydrogen atom or OC(O)R₃₅, wherein R₂₉ to R₃₇ represent, independently of each other, a hydrogen atom, a (C₁-C₆)alkyl, an aryl, or an aryl-(C₁-C₆)alkyl group, notably a hydrogen atom or a (C₁-C₆)alkyl group, typically a hydrogen atom or a (C₁-C₃)alkyl group.

In a preferred embodiment, R₃ represents a hydrogen atom, a halogen atom or a (C₁-C₆)alkyl group, in particular a hydrogen atom, a halogen atom or a (C₁-C₃)alkyl group, preferably a hydrogen atom or a halogen atom.

In the above embodiments, the aryl group, which may be part of an aryl-(C₁-C₆)alkyl group, is preferably a phenyl group.

In the above embodiments, the heterocyclyl group, which may be part of a heterocyclyl-(C₁-C₆)alkyl group, is in particular a 5- or 6-membered, saturated, unsaturated (i.e. not aromatic) or aromatic, notably aromatic, monocyclic group, in which the atoms of the ring comprise one or more, advantageously 1 to 3, heteroatoms selected from O, S and N, preferably O and N, the remainder being carbon atoms, such as a pyridinyl, a pyrimidinyl, a pyrazolyl, a piperazinyl or a piperidinyl group, for instance a a pyridinyl, a pyrimidinyl or a pyrazolyl group.

In the above embodiments, the (C₁-C₆)alkyl group, which may be part of an aryl-(C₁-C₆)alkyl group or a heterocyclyl-(C₁-C₆)alkyl group, is preferably a (C₁-C₃)alkyl group.

According to a particular embodiment of the present invention, R₄ represents a hydrogen atom, a halogen atom, CN, OR₂₉, SR₃₀, NR₃₁R₃₂, a (C₁-C₆)alkyl, a (C₁-C₆)haloalkyl group, an aryl, a heterocyclyl, an aryl-(C₁-C₆)alkyl or a heterocyclyl-(C₁-C₆)alkyl group, wherein said aryl or heterocyclyl group (which may be part of a aryl-(C₁-C₆)alkyl or heterocyclyl-(C₁-C₆)alkyl group) is optionally substituted by one or more substituents, notably one substituent, selected from the group consisting of a halogen atom, CN, NO₂, OR₄₄, SR₄₅, NR₄₆R₄₇, C(O)R₄₈, CO₂R₄₉, OC(O)R₅₀, NR₅₁C(O)R₅₂, C(O)NR₅₃R₅₄, a (C₁-C₆)alkyl and a (C₁-C₆)haloalkyl group, notably a halogen atom, OR₄₄, SR₄₅, NR₄₆R₄₇, C(O)Ra, CO₂R₄₉, C(O)NR₅₃R₅₄, a (C₁-C₆)alkyl and a (C₁-C₆)haloalkyl group, in particular C(O)R₄₈, CO₂R₄₉, C(O)NR₅₃R₅₄, a (C₁-C₆)alkyl and a (C₁-C₆)haloalkyl group, preferably C(O)R₄₈ and a (C₁-C₆)alkyl group.

According to another particular embodiment of the present invention, R₄ represents a hydrogen atom, a halogen atom, CN, OR₂₉, SR₃₀, NR₃₁R₃₂, a (C₁-C₆)alkyl, a (C₁-C₆)haloalkyl group, an aryl, a heterocyclyl, an aryl-(C₁-C₆)alkyl or a heterocyclyl-(C₁-C₆)alkyl group, wherein said aryl or heterocyclyl group is optionally substituted by one or more substituents as defined above.

According to still another particular embodiment of the present invention, R₄ represents a hydrogen atom, a halogen atom, OR₂₉, SR₃₀, NR₃₁R₃₂, a (C₁-C₆)alkyl, an aryl, or a heterocyclyl group, wherein said aryl or heterocyclyl group is optionally substituted by one or more substituents, notably one substituent, selected from the group consisting of a halogen atom, OR₄₄, SR₄₅, NR₄₆R₄₇, C(O)R₄₈, CO₂R₄₉, C(O)NR₅₃R₅₄, a (C₁-C₆)alkyl and a (C₁-C₆)haloalkyl group, in particular C(O)R₄₈, CO₂R₄₉, C(O)NR₅₃R₅₄, a (C₁-C₆)alkyl and a (C₁-C₆)haloalkyl group, preferably C(O)R₄₈ and a (C₁-C₆)alkyl group.

According to yet another particular embodiment of the present invention, R₄ represents a hydrogen atom, a halogen atom, NR₃₁R₃₂, a (C₁-C₆)alkyl, an aryl or a heterocyclyl group, wherein said aryl or heterocyclyl group is optionally substituted by one or more substituents, notably one substituent, selected from the group consisting of C(O)R₄₈, CO₂R₄₉, C(O)NR₅₃R₅₄, a (C₁-C₆)alkyl and a (C₁-C₆)haloalkyl group, preferably C(O)R₄₈ and a (C₁-C₆)alkyl group.

In a preferred embodiment, R₄ represents a hydrogen atom, a halogen atom or a (C₁-C₆)alkyl group, in particular a hydrogen atom or a (C₁-C₃)alkyl group, preferably a hydrogen atom.

In the above embodiments, the aryl group, which may be part of an aryl-(C₁-C₆)alkyl group, is preferably a phenyl group.

In the above embodiments, the heterocyclyl group, which may be part of a heterocyclyl-(C₁-C₆)alkyl group, is in particular a 5- or 6-membered, saturated, unsaturated (i.e. not aromatic) or aromatic, notably saturated, monocyclic group, in which the atoms of the ring comprise one or more, advantageously 1 to 3, heteroatoms selected from O, S and N, preferably O and N, the remainder being carbon atoms, such as a piperazinyl, a piperidinyl, a pyridinyl, a pyrimidinyl or a pyrazolyl group, for instance a piperazinyl or a piperidinyl group.

In the above embodiments, R₂₉ to R₃₂ represent, independently of each other, a hydrogen atom, a (C₁-C₆)alkyl, an aryl or an aryl-(C₁-C₆)alkyl group, said aryl group, which may be part of an aryl-(C₁-C₆)alkyl group, being preferably a phenyl and being optionally substituted by one or more substituents, notably one substituent, selected from the group consisting of a halogen atom, CN, NO₂, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group, advantageously a halogen atom, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group, notably C(O)R₅₉, CO₂R₆₀, C(O)NR₆₄R₆₅, a (C₁-C₆)alkyl and a (C₁-C₆)haloalkyl group, in particular C(O)R₅₉, wherein R₅₅ to R₆₅ represent, independently of each other, a hydrogen atom, a (C₁-C₆)alkyl or an aryl group, notably an aryl group, preferably a phenyl group.

In the above embodiments, R₄₄ to R₅₄ represent, independently of each other, a hydrogen atom, a (C₁-C₆)alkyl or an aryl group, notably an aryl group, preferably a phenyl group.

In the above embodiments, the (C₁-C₆)alkyl group, which may be part of an aryl-(C₁-C₆)alkyl group or a heterocyclyl-(C₁-C₆)alkyl group, is preferably a (C₁-C₃)alkyl group.

According to a particular embodiment of the present invention, R_{4b} represents a hydrogen atom, a halogen atom, OR₂₉, SR₃₀, NR₃₁R₃₂, a (C₁-C₆)alkyl, a (C₁-C₆)haloalkyl group, an aryl, a heterocyclyl, an aryl-(C₁-C₆)alkyl or a heterocyclyl-(C₁-C₆)alkyl group, wherein said aryl or heterocyclyl group (which may be part of a aryl-(C₁-C₆)alkyl or heterocyclyl-(C₁-C₆)alkyl group) is optionally substituted by one or more substituents, notably one substituent, selected from the group consisting of a halogen atom, CN, NO₂, OR₄₄, SR₄₅, NR₄₆R₄₇, C(O)R₄₈, CO₂R₄₉, OC(O)R₅₀, NR₅₁C(O)R₅₂, C(O)NR₅₃R₅₄, a (C₁-C₆)alkyl and a (C₁-C₆)haloalkyl group, notably a halogen atom, OR₄₄, SR₄₅, NR₄₆R₄₇, C(O)R₄₈, CO₂R₄₉, C(O)NR₅₃R₅₄, a (C₁-C₆)alkyl and a (C₁-C₆)haloalkyl group, in particular C(O)R₄₈, CO₂R₄₉, C(O)NR₅₃R₅₄, a (C₁-C₆)alkyl and a (C₁-C₆)haloalkyl group, preferably C(O)R₄₈ and a (C₁-C₆)alkyl group.

According to a particular embodiment of the present invention, R_{4b} represents a hydrogen atom, a halogen atom, OR₂₉, SR₃₀, NR₃₁R₃₂, a (C₁-C₆)alkyl, an aryl, or a heterocyclyl group, wherein said aryl or heterocyclyl group is optionally substituted by one or more substituents, notably one substituent, selected from the group consisting of a halogen atom, OR₄₄, SR₄₅, NR₄₆R₄₇, C(O)R₄₈, CO₂R₄₉, C(O)NR₅₃R₅₄, a (C₁-C₆)alkyl and a (C₁-C₆)haloalkyl group, in particular C(O)R₄₈, CO₂R₄₉, C(O)NR₅₃R₅₄, a (C₁-C₆)alkyl and a (C₁-C₆)haloalkyl group, preferably C(O)R₄₈ and a (C₁-C₆)alkyl group.

According to yet another particular embodiment of the present invention, R_{4b} represents a hydrogen atom, a halogen atom, NR₃₁R₃₂, a (C₁-C₆)alkyl, an aryl or a heterocyclyl group, wherein said aryl or heterocyclyl group is optionally substituted by one or more substituents, notably one substituent, selected from the group consisting of C(O)R₄₈, CO₂R₄₉, C(O)NR₅₃R₅₄, a (C₁-C₆)alkyl and a (C₁-C₆)haloalkyl group, preferably C(O)R₄₈ and a (C₁-C₆)alkyl group.

According to still another particular embodiment of the present invention, R_{4b} represents a hydrogen atom, a halogen atom, a (C₁-C₆)alkyl group, OR₂₉ or NR₃₁R₃₂, preferably a hydrogen atom, a halogen atom, a (C₁-C₃)alkyl group or OR₂₉, more preferably a hydrogen atom.

In a preferred embodiment, R_{4b} represents a hydrogen atom, a halogen atom or a (C₁-C₆)alkyl group, in particular a hydrogen atom or a (C₁-C₃)alkyl group, preferably a hydrogen atom.

In the above embodiments, the aryl group, which may be part of an aryl-(C₁-C₆)alkyl group, is preferably a phenyl group.

In the above embodiments, the heterocyclyl group, which may be part of a heterocyclyl-(C₁-C₆)alkyl group, is in particular a 5- or 6-membered, saturated, unsaturated (i.e. not aromatic) or aromatic, notably saturated, monocyclic group, in which the atoms of the ring comprise one or more, advantageously 1 to 3, heteroatoms selected from O, S and N, preferably O and N, the remainder being carbon atoms.

In the above embodiments, R₂₉ to R₃₂ represent, independently of each other, a hydrogen atom, a (C₁-C₆)alkyl, an aryl or an aryl-(C₁-C₆)alkyl group, said aryl group, which may be part of an aryl-(C₁-C₆)alkyl group, being preferably a phenyl and being optionally substituted by one or more substituents, notably one substituent, selected from the group consisting of a halogen atom, CN, NO₂, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group, advantageously a halogen atom, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group, notably C(O)R₅₉, CO₂R₆₀, C(O)NR₆₄R₆₅, a (C₁-C₆)alkyl and a (C₁-C₆)haloalkyl group, in particular C(O)R₅₉, wherein R₅₅ to R₆₅ represent, independently of each other, a hydrogen atom, a (C₁-C₆)alkyl or an aryl group, notably an aryl group, preferably a phenyl group. Preferably, R₂₉ to R₃₂ represent, independently of each other, a hydrogen atom or a (C₁-C₆)alkyl group, notably a hydrogen atom.

In the above embodiments, R₄₄ to R₅₄ represent, independently of each other, a hydrogen atom, a (C₁-C₆)alkyl or an aryl group, notably an aryl group, preferably a phenyl group.

In the above embodiments, the (C₁-C₆)alkyl group, which may be part of an aryl-(C₁-C₆)alkyl group or a heterocyclyl-(C₁-C₆)alkyl group, is preferably a (C₁-C₃)alkyl group.

In a particular embodiment of the present invention, R₄ is as defined above and R_{4b} represents a hydrogen atom, a halogen atom, OR₂₉ or NR₃₁R₃₂, wherein R₂₉ to R₃₂ represent, independently of each other, a hydrogen atom or a (C₁-C₆)alkyl group, notably a hydrogen atom, preferably R_{4b} represents a hydrogen atom, a halogen atom or OR₂₉ more preferably a hydrogen atom.

In a particular embodiment of the present invention, R₅ represents a hydrogen atom, a halogen atom, CN, OR₂₉, SR₃₀, NR₃₁R₃₂, a (C₁-C₆)alkyl, a (C₁-C₆)haloalkyl group, said alkyl or haloalkyl group being optionally substituted by one or more substituents selected from the group consisting of OR₄₀, SR₄₁ and NR₄₂R₄₃, an aryl, a heterocyclyl, an aryl-(C₁-C₆)alkyl or a heterocyclyl-(C₁-C₆)alkyl group, wherein said aryl or heterocyclyl group (which may be part of a aryl-(C₁-C₆)alkyl or heterocyclyl-(C₁-C₆)alkyl group) is optionally substituted by one or more substituents, notably one substituent, selected from the group consisting of a halogen atom, CN, NO₂, OR₄₄, SR₄₅, NR₄₆R₄₇, C(O)R₄₈, CO₂R₄₉, OC(O)R₅₀, NR₅₁C(O)R₅₂, C(O)NR₅₃R₅₄, a (C₁-C₆)alkyl and a (C₁-C₆)haloalkyl group, notably a halogen atom, OR₄₄, SR₄₅, NR₄₆R₄₇, C(O)R₄₈, CO₂R₄₉, C(O)NR₅₃R₅₄, a (C₁-C₆)alkyl and a (C₁-C₆)haloalkyl group, in particular a halogen atom, OR₄₄, SR₄₅, NR₄₆R₄₇, a (C₁-C₆)alkyl and a (C₁-C₆)haloalkyl group, notably a halogen atom, a (C₁-C₆)alkyl and a (C₁-C₆)haloalkyl group, preferably a (C₁-C₆)alkyl group.

In the above embodiment, R₂₉ to R₃₂ represent, independently of each other, a hydrogen atom, a (C₁-C₆)alkyl, an aryl or an aryl-(C₁-C₆)alkyl group, said aryl group, which may be part of an aryl-(C₁-C₆)alkyl group, being preferably a phenyl and being optionally substituted by one or more substituents, notably one substituent, selected from the group consisting of a halogen atom, CN, NO₂, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group, advantageously a halogen atom, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group, notably C(O)R₅₉, CO₂R₆₀, C(O)NR₆₄R₆₅, a (C₁-C₆)alkyl and a (C₁-C₆)haloalkyl group, in particular C(O)R₅₉, wherein R₅₅ to R₆₅ represent, independently of each other, a hydrogen atom, a (C₁-C₆)alkyl or an aryl group, notably an aryl group, preferably a phenyl group. Preferably, R₂₉ to R₃₂ represent, independently of each other, independently of each other, a hydrogen atom or a (C₁-C₆)alkyl group.

In another particular embodiment of the present invention, R₅ represents a hydrogen atom, a halogen atom, a (C₁-C₆)alkyl, a (C₁-C₆)haloalkyl group, said alkyl or haloalkyl group being optionally substituted by one or more substituents, notably one substituent, selected from the group consisting of OR₄₀, SR₄₁ and NR₄₂R₄₃, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, OR₄₄, SR₄₅, NR₄₆R₄₇, a (C₁-C₆)alkyl and a (C₁-C₆)haloalkyl group, notably a halogen atom, a (C₁-C₆)alkyl and a (C₁-C₆)haloalkyl group, preferably a (C₁-C₆)alkyl group.

In yet another particular embodiment of the present invention, R₅ represents a hydrogen atom, a halogen atom, a (C₁-C₆)alkyl, a (C₁-C₆)haloalkyl group or a heterocyclyl-(C₁-C₆)alkyl group, said alkyl or haloalkyl group being optionally substituted by OR₄₀, and said heterocyclyl being optionally substituted by one or more (C₁-C₆)alkyl group.

In a preferred embodiment, R₅ represents a hydrogen atom, a halogen atom or a (C₁-C₆)alkyl group, in particular a hydrogen atom or a (C₁-C₃)alkyl group.

Preferably, R₅ represents a hydrogen atom.

In the above embodiments, the aryl group, which may be part of an aryl-(C₁-C₆)alkyl group, is preferably a phenyl group.

In the above embodiments, the heterocyclyl group, which may be part of a heterocyclyl-(C₁-C₆)alkyl group, is in particular a 5- or 6-membered, saturated, unsaturated (i.e. not aromatic) or aromatic, notably saturated, monocyclic group, in which the atoms of the ring comprise one or more, advantageously 1 to 3, heteroatoms selected from O, S and N, preferably O and N, the remainder being carbon atoms, such as a piperazinyl group.

In the above embodiments, R₄₀ to R₄₃ represent, independently of each other, a hydrogen atom or a (C₁-C₆)alkyl group, notably a hydrogen atom.

In the above embodiments, R₄₄ to R₅₄ represent, independently of each other, a hydrogen atom, a (C₁-C₆)alkyl or an aryl group, notably an aryl group, preferably a phenyl group.

In the above embodiments, the (C₁-C₆)alkyl group, which may be part of an aryl-(C₁-C₆)alkyl group or a heterocyclyl-(C₁-C₆)alkyl group, is preferably a (C₁-C₃)alkyl group.

In a particular embodiment of the present invention, R₆ represents a hydrogen atom, a (C₁-C₃)alkyl, an aryl-(C₁-C₃)alkyl or a —CH₂—CH₂—O—CH₂—CH₂—NH₂ group, or a (C₁-C₆)alkylcarbonyl group optionally substituted with one or more substituents selected from the group consisting of OH, SH, NH₂, a (C₁-C₃)alkoxy, a (C₁-C₃)thioalkoxy and a (C₁-C₃)alkylamino group, preferably Rr represents a hydrogen atom, a methyl, an ethyl, a benzyl, a —CH₂—CH₂—O—CH₂—CH₂—NH₂ or a (C₁-C₆)alkylcarbonyl group optionally substituted with one or more substituents selected from the group consisting of OH, NH₂ and SH, in particular R₆ represents a hydrogen atom, a —CH₂—CH₂—O—CH₂—CH₂—NH₂ or an ethyl group, notably an ethyl group.

In a preferred embodiment, R₆ represents a hydrogen atom, a (C₁-C₃)alkyl or a —CH₂—CH₂—O—CH₂—CH₂—NH₂ group, in particular a hydrogen atom or a —CH₂—CH₂—O—CH₂—CH₂—NH₂ group.

In a first aspect of the present invention,

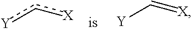

X is N, Y is N(R₂) and Z is C(H), and the inhibitor of a pharmaceutical composition according to the invention is thus of the following general formula (Li):

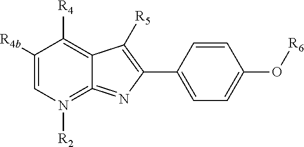

wherein R₂, R₄, R_{4b}, R₅ and R₆ are as defined in any one of the above embodiments.

In particular, R₆ represents a hydrogen atom or a (C₁-C₆)alkyl group, preferably a (C₁-C₃)alkyl group, notably a methyl or an ethyl group, advantageously R₄ represents an ethyl group.

In a second aspect of the present invention,

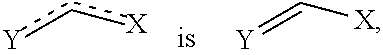

X is N(R₁), Y is N and Z is C(R₃), and the inhibitor of a pharmaceutical composition according to the invention is thus of the following general formula (I.ii.a):

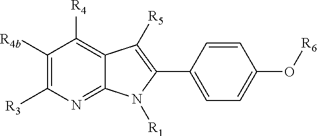

wherein R₁, R₃, R₄, R_{4b}, R₅ and R₆ are as defined in any one of the above embodiments.

In particular, R₆ represents a hydrogen atom, a (C₁-C₃)alkyl such as an ethyl, a —CH₂—CH₂—O—CH₂—CH₂—NH₂ or a (C₁-C₆)alkylcarbonyl group optionally substituted with one or more substituents selected from the group consisting of OH, NH₂ and SH, advantageously R₆ represents a hydrogen atom, a —CH₂—CH₂—O—CH₂—CH₂—NH₂ or a (C₁-C₃)alkyl such as an ethyl group.

In a third aspect of the present invention,

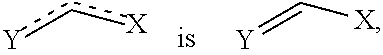

X is N(R₁), Y is N⁺(O⁻) and Z is C(R₃), and the inhibitor of a pharmaceutical composition according to the invention is thus of the following general formula (I.ii.b):

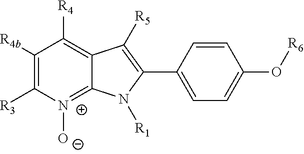

wherein R₁, R₃, R₄, R_{4b}, R₅ and R₆ are as defined in any one of the above embodiments.

In particular, R₆ represents a hydrogen atom or a (C₁-C₆)alkyl group, preferably a (C₁-C₃)alkyl group, notably a methyl or an ethyl group, advantageously R₆ represents an ethyl group.

In a fourth aspect of the present invention,

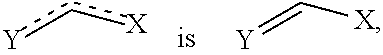

X is N(R₁), Y is CH and Z is N, and the inhibitor of a pharmaceutical composition according to the invention is thus of the following general formula (I.ii.c):

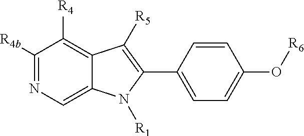

wherein R₁, R₄, R_{4b}, R₅ and R₆ are as defined in any one of the above embodiments.

In particular, R₆ represents a hydrogen atom or a (C₁-C₆)alkyl group, preferably a (C₁-C₃)alkyl group, notably a methyl or an ethyl group, advantageously R₆ represents an ethyl group.

In a fifth aspect of the present invention,

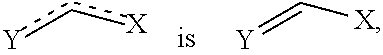

X is N(R₁) and Y and Z are CH, and the inhibitor of a pharmaceutical composition according to the invention is thus of the following general formula (I.ii.d):

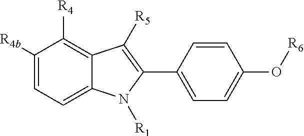

wherein R₁, R₄, R_{4b}, R₅ and R₆ are as defined in any one of the above embodiments.

In particular, R₆ represents a hydrogen atom, a (C₁-C₆)alkyl group or an aryl-(C₁-C₆)alkyl group, preferably a hydrogen atom, a (C₁-C₃)alkyl group or an aryl-(C₁-C₃)alkyl group, notably a hydrogen atom, a methyl, an ethyl or a benzyl group, advantageously R₆ represents a hydrogen atom, a methyl or a benzyl group, typically a hydrogen atom.

In a preferred embodiment, the inhibitor is a compound of the following general formula (I.iii):

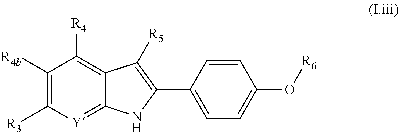

wherein R₃, R₄, R_{4b}, R₅ and R₆ are as defined in any one of the above embodiments, and wherein Y′ is N or CH.

In particular:
R₃, R₄, R_{4b} and R₅ represent, independently of each other, a hydrogen atom, a halogen atom or a (C₁-C₆)alkyl group, in particular a hydrogen atom, a halogen atom or a (C₁-C₃)alkyl group, preferably a hydrogen atom or a halogen atom, and
R₆ represents a hydrogen atom, a (C₁-C₃)alkyl or a —CH₂—CH₂—O—CH₂—CH₂—NH₂ group, in particular a hydrogen atom or a —CH₂—CH₂—O—CH₂—CH₂—NH₂ group.

The inhibitor of a pharmaceutical composition according to the invention may notably be selected from the group consisting of compounds 1 to 45, represented below, and the pharmaceutically acceptable salts and/or solvates thereof.

[chemical structure]

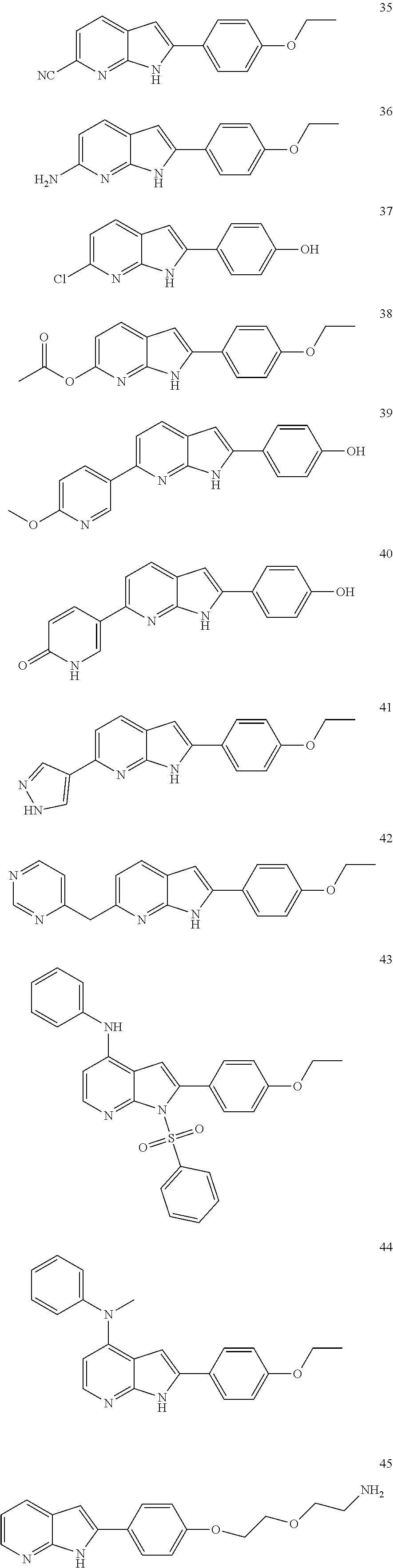

In particular, the inhibitor of a pharmaceutical composition according to the invention may be selected from the group consisting of compounds 1, 7, 37, 45 and the pharmaceutically acceptable salts and/or solvates thereof.

### Nigratine and Derivatives Thereof

In a particular embodiment, the inhibitor of a pharmaceutical composition according to the invention is nigratine or a derivative thereof.

In this particular embodiment, the inhibitor is preferably a compound of the following general formula (I):

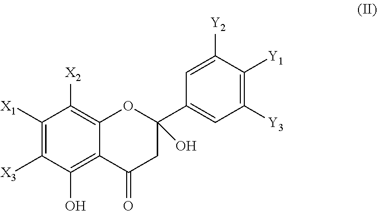

or a pharmaceutically acceptable salt and/or solvate thereof, wherein:
X₁, X₂ and X₃ represent, independently of each other, a hydrogen atom, a (C₁-C₆)alkyl, an aryl, an aryl-(C₁-C₆)alkyl or an OH group, or a group selected from ORₓ, SRₓ, SO₂Rₓ and NRₓR_{z},
   wherein at least one of X₁, X₂ and X₃ represents a (C₁-C₆)alkyl, an aryl or an aryl-(C₁-C₆)alkyl group, or a group selected from ORₓ, SRₓ, SO₂Rₓ and NRₓR_{z}, wherein
   Rₓ a (C₁-C₆)alkyl, an aryl or an aryl-(C₁-C₆)alkyl group,
   R_{z} is a hydrogen atom or a (C₁-C₆)alkyl group, and
   the aryl groups are optionally substituted with one or several groups selected from a halogen atom, —OR₆₆, —NR₆₇R₆₈, —SR₆₉, —S(O)R₇₀, —SO₂R₇₁, —OCOR₇₂, —CO₂R₇₃, —CONR₇₄R₇₅, —CO₂R₇₆, nitro (—NO₂) and cyano (—CN);
Y₁, Y₂ and Y₃ represent, independently of each other, a hydrogen atom, a (C₁-C₆)alkyl, an aryl, an aryl-(C₁-C₆)alkyl or an OH group, or a group selected from OR_{y}, SR_{y}, SO₂R_{y} and NR_{y}R′_{z},
   wherein at least one of Y₁, Y₂ and Y₃ represents a (C₁-C₆)alkyl, an aryl or an aryl-(C₁-C₆)alkyl group, or a group selected from OR_{y}, SR_{y}, SO₂R_{y} and NR_{y}R′_{z}, wherein
   R_{y} is a (C₁-C₆)alkyl, an aryl or an aryl-(C₁-C₆)alkyl group,
   R′_{z} is a hydrogen atom or a (C₁-C₆)alkyl group, and the aryl groups are optionally substituted with one or several groups selected from a halogen atom, —OR₆₆, —NR₆₇R₆₈, —SR₆₉, —S(O)R₇₀, —SO₂R₇₁, —OCOR₇₂, —CO₂R₇₃, —CONR₇₄R₇₅, —CO₂R₇₆, nitro (—NO₂) and cyano (—CN); and
R₆₆ to R₇₇ are, independently of one another, a hydrogen atom or a (C₁-C₆)alkyl group.

According to a particular embodiment of the present invention, X₁, X₂ and X₃ represent, independently of each other, a hydrogen atom, or a group selected from ORₓ, SRₓ, SO₂Rₓ and NRₓR_{z}, wherein at least one of X₁, X₂ and X₃ is not a hydrogen atom.

In another particular embodiment of the present invention, X₁, X₂ and X₃ represent, independently of each other, a hydrogen atom, a (C₁-C₆)alkyl, an aryl, an aryl-(C₁-C₆)alkyl group, an OH or an ORₓ group, wherein at least one of X₁, X₂ and X₃ represents an ORₓ group.

In still another particular embodiment of the present invention, X₁, X₂ and X₃ represent, independently of each other, a hydrogen atom or an ORₓ group, wherein at least one of X₁, X₂ and X₃ represents an ORₓ group.

In the above embodiments, Rₓ is preferably a (C₁-C₆)alkyl group, notably a (C₁-C₃)alkyl group such as methyl, ethyl, n-propyl, more preferably methyl.

In the above embodiments, the aryl groups are optionally substituted with one or several groups selected from a halogen atom, —OR₆₆, —NR₆₇R₆₈, —SR₆₉, —S(O)R₇₀, —SO₂R₇₁, —OCOR₇₂, —CO₂R₇₃, —CONR₇₄R₇₅, —CO₂R₇₆, nitro (—NO₂) and cyano (—CN).

In another embodiment, X₁ represents a (C₁-C₆)alkyl, an aryl or an aryl-(C₁-C₆)alkyl group, or a group selected from ORₓ, SRₓ, SO₂Rₓ and NRₓR_{z}, wherein Rₓ is selected from a (C₁-C₆)alkyl, an aryl and an aryl-(C₁-C₆)alkyl group, Rₓ being preferably a (C₁-C₆)alkyl group, notably a (C₁-C₃)alkyl group such as methyl, ethyl, n-propyl, more preferably methyl; and X₂ and X₃ represent, independently of each other, a hydrogen atom or a (C₁-C₆)alkyl group, preferably a hydrogen atom.

In still another embodiment, X₁ represents a group selected from ORₓ, SRₓ, SO₂Rₓ and NRₓR_{z}, wherein Rₓ is selected from a (C₁-C₆)alkyl, an aryl and an aryl-(C₁-C₆)alkyl group, Rₓ being preferably a (C₁-C₆)alkyl group, notably a (C₁-C₃)alkyl group such as methyl, ethyl, n-propyl, more preferably methyl; and X₂ and X₃ represent, independently of each other, a hydrogen atom or a (C₁-C₆)alkyl group, preferably a hydrogen atom.

In a preferred embodiment, X₁ represents an ORₓ group, Rₓ being advantageously a (C₁-C₆)alkyl group.

In another preferred embodiment, X₂ and X₃ each represent a hydrogen atom.

In a yet another preferred embodiment, X₁ represents an ORₓ group, wherein Rₓ is selected from a (C₁-C₆)alkyl, an aryl and an aryl-(C₁-C₆)alkyl group, Rₓ being advantageously a (C₁-C₆)alkyl group, notably a (C₁-C₃)alkyl group such as methyl, ethyl, n-propyl, more advantageously methyl; and X₂ and X₃ represent, independently of each other, a hydrogen atom or a (C₁-C₆)alkyl group, advantageously a hydrogen atom.

In the above embodiments, the aryl groups are optionally substituted with one or several groups selected from a halogen atom, —OR₆₆, —NR₆₇R₆₈, —SR₆₉, —S(O)R₇₀, —SO₂R₇₁, —OCOR₇₂, —CO₂R₇₃, —CONR₇₄R₇₅, —CO₂R₇₆, nitro (—NO₂) and cyano (—CN).

According to a particular embodiment of the present invention, Y₁, Y₂ and Y₃ represent, independently of each other, a hydrogen atom, or a group selected from OR_{y}, SR_{y}, SO₂R_{y} and NR_{y}R′_{z}, wherein at least one of Y₁, Y₂ and Y₃ is not a hydrogen atom.

In another particular embodiment of the present invention, Y₁, Y₂ and Y₃ represent, independently of each other, a hydrogen atom, a (C₁-C₆)alkyl, an aryl, an aryl-(C₁-C₆)alkyl group, an OH, or an OR_{y} group, wherein at least one of Y₁, Y₂ and Y₃ represents an OR_{y} group.

In still another particular embodiment of the present invention, Y₁, Y₂ and Y₃ represent, independently of each other, a hydrogen atom or an OR_{y} group, wherein at least one of Y₁, Y₂ and Y₃ represents an OR_{y} group.

In the above embodiments, R_{y} is preferably a —(C₁-C₆)alkyl-aryl group, such as benzyl or —CH₃-naphtyl, more preferably benzyl.

In another embodiment, Y₁ represents a (C₁-C₆)alkyl, an aryl or an aryl-(C₁-C₆)alkyl group, or a group selected from OR_{y}, SR_{y}, SO₂R_{y} and NR_{y}R′_{z}, wherein R_{y} is selected from a (C₁-C₆)alkyl, an aryl and an aryl-(C₁-C₆)alkyl group, R_{y} being preferably a —(C₁-C₆)alkyl-aryl group, such as benzyl or —CH₃-naphtyl, more preferably benzyl; and Y₂ and Y₃ represent, independently of each other, a hydrogen atom or a (C₁-C₆)alkyl group, preferably a hydrogen atom.

In still another embodiment, Y₁ represents a group selected from OR_{y}, SR_{y}, SO₂R_{y} and NR_{y}R′_{z}, wherein R_{y} is selected from a (C₁-C₆)alkyl, an aryl and an aryl-(C₁-C₆)alkyl group, R_{y} being preferably —(C₁-C₆)alkyl-aryl group, such as benzyl or —CH₃-naphtyl, more preferably benzyl, and Y₂ and Y₃ represent, independently of each other, a hydrogen atom or a (C₁-C₆)alkyl group, preferably a hydrogen atom.

In a preferred embodiment, Y₁ represents an OR_{y} group, wherein R_{Y} is selected from a (C₁-C₆)alkyl, an aryl and an aryl-(C₁-C₆)alkyl group, R_{y} being advantageously a —(C₁-C₆)alkyl-aryl group, such as benzyl or —CH₃-naphtyl, more preferably benzyl; and Y₂ and Y₃ represent, independently of each other, a hydrogen atom or a (C₁-C₆)alkyl group, advantageously a hydrogen atom.

In the above embodiments, the aryl groups are optionally substituted with one or several groups selected from a halogen atom, —OR₆₆, —NR₆₇R₆₈, —SR₆₉, —S(O)R₇₀, —SO₂R₇₁, —OCOR₇₂, —CO₂R₇₃, —CONR₇₄R₇₅, —CO₂R₇₆ nitro (—NO₂) and cyano (—CN).

According to a particular embodiment of the present invention:
X₁, X₂ and X₃ represent, independently of each other, a hydrogen atom, a (C₁-C₆)alkyl, an aryl, an aryl-(C₁-C₆)alkyl or an ORₓ group, wherein at least one of X₁, X₂ and X₃ is not a hydrogen atom, and wherein Rₓ is selected from a (C₁-C₆)alkyl, an aryl and an aryl-(C₁-C₆)alkyl group; and
Y₁, Y₂ and Y₃ represent, independently of each other, a hydrogen atom, a (C₁-C₆)alkyl, an aryl, an aryl-(C₁-C₆)alkyl group, an OH or an OR_{y} group, wherein at least one of Y₁, Y₂ and Y₃ represents a (C₁-C₆)alkyl, an aryl, an aryl-(C₁-C₆)alkyl group or an OR_{y} group, wherein R_{y} is selected from a (C₁-C₆)alkyl, an aryl and an aryl-(C₁-C₆)alkyl group.

According to another particular embodiment:
X₁ represents a (C₁-C₆)alkyl, an aryl, an aryl-(C₁-C₆)alkyl or an ORₓ group, wherein Rₓ is a (C₁-C₆)alkyl, an aryl or an aryl-(C₁-C₆)alkyl group;
X₂ and X₃ represent, independently of each other, a hydrogen atom or a (C₁-C₆)alkyl group; and
Y₁, Y₂ and Y₃ represent, independently of each other, a hydrogen atom, a (C₁-C₆)alkyl, an aryl, an aryl-(C₁-C₆)alkyl, an OH or an OR_{y} group, wherein at least one of Y₁, Y₂ and Y₃ represents a (C₁-C₆)alkyl, an aryl, an aryl-(C₁-C₆)alkyl or an OR_{y} group, wherein R_{y} is selected from a (C₁-C₆)alkyl, an aryl and an aryl-(C₁-C₆)alkyl group.

According to yet another particular embodiment:
X₁ represents an ORₓ group, wherein Rₓ is selected from a (C₁-C₆)alkyl, an aryl and an aryl-(C₁-C₆)alkyl group, Rₓ being advantageously a (C₁-C₆)alkyl group, notably a (C₁-C₃)alkyl group such as methyl, ethyl, n-propyl, more advantageously methyl;
X₂ and X₃ represent, independently of each other, a hydrogen atom or a (C₁-C₆)alkyl group, advantageously a hydrogen atom;
Y₁ represents an OR_{y} group, wherein R_{y} is selected from a (C₁-C₆)alkyl, an aryl and an aryl-(C₁-C₆)alkyl group, R_{y} being advantageously a —(C₁-C₆)alkyl-aryl group, such as benzyl or —CH₃-naphtyl, more preferably benzyl; and
Y₂ and Y₃ each represent, independently of each other, a hydrogen atom or a (C₁-C₆)alkyl group, advantageously a hydrogen atom.

According to a preferred embodiment, the inhibitor of a pharmaceutical composition according to the invention is of the following formula (II.i):

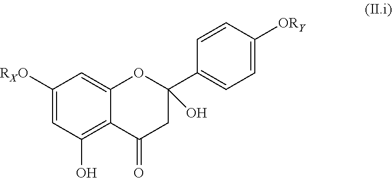

or a pharmaceutically acceptable salt and/or solvate thereof, wherein:
Rₓ represents a (C₁-C₆)alkyl group, notably a (C₁-C₃)alkyl group such as methyl, ethyl, n-propyl, more advantageously methyl, and
R_{y} represents an aryl-(C₁-C₆)alkyl group, such as benzyl or —CH₃-naphtyl, more preferably benzyl.

The inhibitor of a pharmaceutical composition according to the invention may notably be selected from the group consisting of compounds 46 to 49, represented below, and the pharmaceutically acceptable salts and/or solvates thereof.

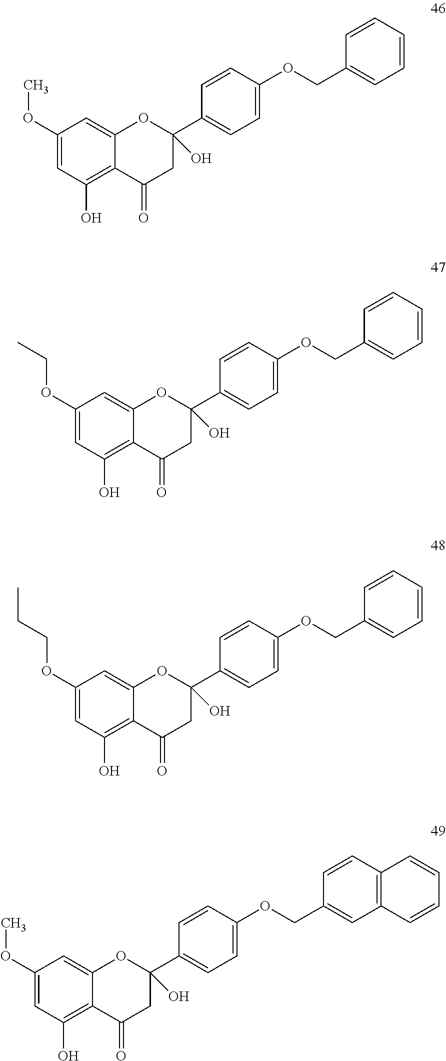 [SMILES: COc1cc(O)c2c(c1)OC(O)(c1ccc(OCc3ccccc3)cc1)CC2=O ; CCOc1cc(O)c2c(c1)OC(O)(c1ccc(OCc3ccccc3)cc1)CC2=O ; CCCOc1cc(O)c2c(c1)OC(O)(c1ccc(OCc3ccccc3)cc1)CC2=O ; COc1cc(O)c2c(c1)OC(O)(c1ccc(OCc3ccc4ccccc4c3)cc1)CC2=O]

In particular, the inhibitor of a pharmaceutical composition according to the invention is compound 46 or a pharmaceutically acceptable salt and/or solvate thereof.

In particular, the inhibitor of a pharmaceutical composition according to the invention may be selected from the group consisting of compounds 1, 7, 37, 45, 46 and the pharmaceutically acceptable salts and/or solvates thereof.

### Pharmaceutical Composition

The pharmaceutical composition according to the invention comprises a combination of N-acetylcysteine (NAC) or a pharmaceutical salt or a derivative thereof with at least one inhibitor of regulated necrotic cell death, such as necroptosis and/or ferroptosis.

In a particular embodiment, the inhibitor of regulated necrotic cell death is a ferroptosis inhibitor, preferably a ferroptosis and necroptosis inhibitor. Said inhibitor may be, in particular, sibiriline, nigratine or a derivative thereof, as defined above.

In this particular embodiment, the NAC/inhibitor molar ratio is advantageously comprised between 500:1 and 1:1, preferably between 200:1 and 1:1, notably between 100:1 and 1:1, in particular between 50:1 and 1:1, for example between 20:1 and 1:1.

In a preferred embodiment, the inhibitor of regulated necrotic cell death is sibiriline (compound 7) and the NAC/inhibitor weight ratio is advantageously comprised between 100:1 and 1:1, in particular between 50:1 and 1:1, notably between 30:1 and 1:1, for example 20:1.

In a particular embodiment, the or N-acetylcysteine amide (NACA)/inhibitor molar ratio is advantageously comprised between 200:1 and 1:1, preferably between 100:1 and 1:1, notably between 50:1 and 1:1, in particular between 30:1 and 1:1, for example between 20:1 and 1:1.

In a particular embodiment, the or N-acetylcysteine ethyl ester (NACET/inhibitor molar ratio is advantageously comprised between 500:1 and 1:1, preferably between 200:1 and 1:1, notably between 100:1 and 1:1, in particular between 50:1 and 1:1, for example between 20:1 and 1:1.

The pharmaceutical composition according to the invention may further comprise at least one pharmaceutically acceptable excipient.

The term “pharmaceutically acceptable excipient” is intended to mean, in the framework of the present invention, a substance which is pharmaceutically acceptable, as defined above, formulated alongside the active ingredients of the pharmaceutical composition, included for the purpose of long-term stabilization, bulking up solid formulations that contain potent active ingredients in small amounts, to confer a therapeutic improvement on the active ingredient in the final dosage form (such as facilitating drug absorption, reducing viscosity, or enhancing solubility), or to enhance the taste or the appearance of the pharmaceutical composition. The appropriate excipients can be easily and wisely selected by the skilled person, taking into account notably the dosage form and the route of administration.

The pharmaceutical compositions according to the invention may be formulated notably for oral administration, for topical administration or for injection, wherein said compositions are intended for mammals, including humans.

The pharmaceutical composition can be administered orally in a solid or liquid (solution or suspension) form.

A solid composition can be in the form of tablets, gelatin capsules, powders, granules and the like. When a solid composition is prepared in the form of tablets, the active ingredients are mixed with a pharmaceutical vehicle such as gelatin, starch, lactose, magnesium stearate, talc, gum arabic and the like. The tablets may be coated with sucrose or with other suitable materials, or they may be treated in such a way that they have a prolonged or delayed activity and they continuously release a predetermined amount of active principle. In powders or granules, the active ingredients can be mixed or granulated with dispersing agents, wetting agents or suspending agents and with flavor correctors or sweeteners. In gelatin capsules, the active ingredients can be introduced into soft or hard gelatin capsules in the form of a powder or granules such as mentioned previously or in the form of a liquid composition such as mentioned below.

A liquid composition can contain the active ingredients together with a sweetener, a taste enhancer or a suitable coloring agent in a solvent such as water. The liquid composition can also be obtained by suspending or dissolving a powder or granules, as mentioned above, in a liquid such as water, juice, milk, etc. It can be for example a syrup or an elixir.

For topical administration, the pharmaceutical composition may be in any form allowing an application to the surface of the skin or mucous membranes: cream, gel, ointment, patch, etc.

For administration by injection, aqueous suspensions, isotonic saline solutions or sterile and injectable solutions which contain pharmacologically compatible dispersing agents and/or wetting agents are used.

The inhibitor of the pharmaceutical composition may be used in doses ranging between 0.01 mg and 2,000 mg per day, given in a single dose once per day or administered in several doses throughout the day, for example twice a day in equal doses. The dose administered per day is advantageously between 5 mg and 500 mg, even more advantageously between 10 mg and 200 mg. The effective dose of the inhibitor can be determined by one skilled in the art by routine tests including assessment of the effect of administration of the inhibitor on the disorders which are sought to be prevented and/or treated by said administration. For example, such tests can be implemented by analyzing both quantitative and qualitative effect of the administration of different amounts of the inhibitor on a set of markers (biological and/or clinical) characteristics of said disorder, in particular from a biological sample of a person. Besides, as is well-known to the skilled person, the suitable dose and the associated dosing regimen for treating a given disease in a given patient will depend on several other factors, such as the stage of the disease as well as the physical and medical condition of the patient.

N-acetylcysteine (NAC), N-acetylcysteine amide or N-acetylcysteine ethyl ester or N-acetylcysteine methyl ester may be used in doses ranging between 50 mg and 5,000 mg per day, given in a single dose once per day or administered in several doses throughout the day, for example twice or three times a day in equal doses. The dose administered per day is advantageously between 100 mg and 2000 mg, even more advantageously between 500 mg and 1500 mg.

### Applications

The present invention is also directed to a pharmaceutical composition as defined above, for preventing and/or treating a disorder associated with regulated necrotic cell death, such as necroptosis and/or ferroptosis.

In a particular embodiment, said disorder is associated with ferroptosis, notably with both ferroptosis and necroptosis.

The disorder associated with ferroptosis may be myocardial ischemia-reperfusion injury, notably occurring after artery ligation or myocardial necrosis in myocardial infarction; cardiomyopathy, notably doxorubicin-induced cardiomyopathy; strokes, notably ischemic stroke or hemorrhagic stroke; cardiovascular disease, such as aortic dissection; traumatic brain injury; contusion spinal cord injury; neurodegenerative disorders, in particular chronic neurodegenerative disorders, more particularly Alzheimer's disease, Huntington's disease, Parkinson's disease, amyotrophic lateral sclerosis (Charcot's disease), multiple sclerosis, Friedreich's ataxia and dementia; vision loss, in particular due to retinal detachment or cataract; retinal disorders, notably Stargardt disease or age-related macular degeneration (AMD), in particular dry AMD; chronic liver diseases, notably non-alcoholic steatohepatitis (NASH), chronic infections such as hepatitis, and alcoholic cirrhosis; acute liver injury and acute liver failure, notably resulting from a drug-induced liver injury (DILI), such as acetaminophen (APAP)-induced liver injury, from an ischemia-reperfusion injury induced by a septic or hemorrhagic shock, from fulminant viral hepatitis, from auto-immune origin or from alcohol intake; skin inflammatory diseases, such as psoriasis; toxic epidermal necrolysis (Lyell syndrome); acute kidney injury (AKI) or acute renal failure, such as oxalate-, folic acid (FA)- and cisplatin-induced AKI, renal ischemia-reperfusion injury and acute tubular necrosis; chronic obstructive pulmonary disease (COPD); bronchial asthma; lung injury caused by a bacterial infection, notably by Pseudomonas aeruginosa or Mycobacterium tuberculosis; pulmonary fibrosis, such as radiation induced-lung fibrosis (RILF) and paraquat-induced pulmonary damage; necrotizing enterocolitis; inflammatory bowel diseases, such as Crohn's disease and ulcerative colitis; haemochromatosis; β-thalassemia; hemolytic disorders; cytokinic storm during a viral infection; radiation-induced necrosis; rheumatoid arthritis; type I diabetes; insulin resistance related to obesity; epilepsy, including mitochondrial disease-related epilepsy and intractable epilepsy; and pathologies related to stress-induced premature tissue senescence, such as atherosclerosis, hypertension and type II diabetes.

Preferably, the disorder associated with ferroptosis is selected from the group consisting of myocardial ischemia-reperfusion injury, notably occurring after artery ligation or myocardial necrosis in myocardial infarction; strokes, notably ischemic stroke or hemorrhagic stroke; traumatic brain injury; neurodegenerative disorders, in particular chronic neurodegenerative disorders, more particularly Alzheimer's disease, Huntington's disease, Parkinson's disease, amyotrophic lateral sclerosis (Charcot's disease) and multiple sclerosis; vision loss, in particular due to retinal detachment or cataract; retinal disorders, notably Stargardt disease or age-related macular degeneration (AMD), in particular dry AMD; chronic liver diseases, notably non-alcoholic steatohepatitis (NASH); acute liver injury and acute liver failure, notably resulting from a drug-induced liver injury (DILI), such as acetaminophen (APAP)-induced liver injury, or from an ischemia-reperfusion injury induced by a septic or hemorrhagic shock; and acute kidney injury (AKI) or acute renal failure, such as folic acid (FA)-induced AKI, cisplatin-induced AKI, renal ischemia-reperfusion injury and acute tubular necrosis.

In particular, the disorder associated with ferroptosis is selected from the group consisting of neurodegenerative disorders, in particular chronic neurodegenerative disorders, more particularly Alzheimer's disease, Huntington's disease, Parkinson's disease, amyotrophic lateral sclerosis (Charcot's disease) and multiple sclerosis; vision loss, in particular due to retinal detachment or cataract; retinal disorders, notably Stargardt disease or age-related macular degeneration (AMD), in particular dry AMD; acute liver injury and acute liver failure, notably resulting from a drug-induced liver injury (DILI), such as acetaminophen (APAP)-induced liver injury, or from an ischemia-reperfusion injury induced by a septic or hemorrhagic shock; and acute kidney injury (AKI) or acute renal failure, such as folic acid (FA)-induced AKI and cisplatin-induced AKI.

In particular, disorder associated with regulated necrotic cell death is a disorder associated with ferroptosis and necroptosis and can be selected from the group consisting of: brain diseases or disorders including neurodegenerative diseases or disorders (notably Alzheimer's disease, Huntington's disease, Parkinson's disease, amyotrophic lateral sclerosis (Charcot's disease), dementia, Friedreich's ataxia and multiple sclerosis), stroke (notably ischemic stroke and hemorrhagic stroke), traumatic brain injury, epilepsy; eye diseases or disorders including retinopathy, degenerative eye diseases or disorders such as retinal degenerative diseases or disorders (notably Stargardt disease and age-related macular degeneration (AMD)); infectious diseases including diseases caused by virus or bacteria (including lung injury caused by a bacterial infection, cytokinic storm during a viral infection); autoimmune diseases including psoriasis and rheumatoid arthritis; inflammatory diseases including chronic liver diseases (notably non-alcoholic steatohepatitis (NASH)), toxic epidermal necrolysis (Lyell's syndrome), bronchial asthma, pulmonary fibrosis, necrotizing enterocolitis, inflammatory bowel diseases (such as Crohn's disease), type I and II diabetes, haemochromatosis, atherosclerosis, insulin resistance related to obesity; pathologies related to stress-induced premature tissue senescence including age-related disorders such as atherosclerosis, hypertension and type II diabetes; liver injury including acute liver failure (notably resulting from a drug-induced liver injury (DILI), such as acetaminophen (APAP)-induced liver injury, or from an ischemia-reperfusion injury induced by a septic or hemorrhagic shock) and chronic liver diseases; hypertension; haemochromatosis; hemolytic disorders; ischemic disorders affecting the heart, brain, or kidneys; kidney injury including acute kidney injury (AKI, also known as acute renal failure (ARF)) such as folic acid (FA)-induced AKI, cisplatin-induced AKI, renal ischemia-reperfusion injury, acute tubular necrosis, liver fibrosis; heart injury including myocardial infarction, myocardial ischemia-reperfusion injury, notably occurring after artery ligation or myocardial necrosis in myocardial infarction; aortic aneurysm; pancreatitis; radiation-induced necrosis; chronic obstructive pulmonary disease, acute respiratory distress disorder; diseases related to transplantation; cancers including liver cancers (notably hepatocellular carcinoma), eye cancers, gastric cancers, colorectal cancers, pancreatic cancers, brain cancers, lung cancers, adrenocortical carcinomas, kidney cancers (notably clear cell renal cell carcinomas).

More particularly, disorder associated with regulated necrotic cell death is a disorder associated with ferroptosis and necroptosis and can be selected from the group consisting of: brain diseases or disorders including neurodegenerative diseases or disorders, stroke, traumatic brain injury, epilepsy; eye diseases or disorders including retinopathy, degenerative eye diseases or disorders; infectious diseases; autoimmune diseases; inflammatory diseases; pathologies related to stress-induced premature tissue senescence; liver injury including acute liver failure and chronic liver diseases; hypertension; haemochromatosis; hemolytic disorders; ischemic disorders affecting the heart, brain, or kidneys; kidney injury including acute kidney injury, renal ischemia-reperfusion injury, acute tubular necrosis, liver fibrosis; heart injury; aortic aneurysm; pancreatitis; radiation-induced necrosis; chronic obstructive pulmonary disease, acute respiratory distress disorder; diseases related to transplantation; cancers including liver cancers, eye cancers, brain cancers, kidney cancers.

In another embodiment, disorder associated with regulated necrotic cell death is a disorder associated with ferroptosis and necroptosis and can be selected from the group consisting of: brain diseases or disorders including neurodegenerative diseases or disorders, stroke, traumatic brain injury; eye diseases and disorders including retinopathy; liver diseases or disorders including acute liver injury such as acute liver failure; kidney diseases or disorders including acute kidney injury; diseases related to transplantation; cancers including liver cancers (such as hepatocellular carcinoma), eye cancer, kidney cancers (such as clear cell renal cell carcinomas).

The disorder associated with both ferroptosis and necroptosis may be acute liver injury and acute liver failure, acute renal failure or acute kidney injury (AKI), acute tubular necrosis, radiation-induced necrosis, ischemic disorders affecting the heart, brain, or kidneys, rheumatoid arthritis, psoriasis, neurodegenerative diseases such as Alzheimer's disease, Parkinson's diseases, lateral amyotrophic sclerosis (Charcot's disease) and dementia, dry (atrophic) age-related macular degeneration (AMD), haemochromatosis, necrotizing enterocolitis, non-alcoholic steatohepatitis (NASH), Friedreich's ataxia, inflammatory bowel diseases such as Crohn's disease, toxic epidermal necrolysis (Lyell's syndrome), type I diabetes, and diseases related to stress-induced premature tissue senescence, including age-related disorders such as atherosclerosis, hypertension and type II diabetes.

The present invention also relates to a method for inhibiting regulated necrotic cell death, in particular for inhibiting ferroptosis, more particularly for inhibiting both ferroptosis and necroptosis, comprising the administration to a person in need thereof of an effective dose of a pharmaceutical composition as defined above. In particular, the present invention relates to a method for preventing and/or treating a disorder associated with regulated necrotic cell death, such as necroptosis and/or ferroptosis, comprising the administration to a person in need thereof of an effective dose of a pharmaceutical composition as defined above.

In a particular embodiment, said disorder is associated with ferroptosis, notably with both ferroptosis and necroptosis, and is as defined above.

The present invention also relates to a method for inhibiting lysosomal permeabilization and/or lysosome-dependent cell death, in particular in a subject suffering from a neurodegenerative disease, comprising the administration to a person in need thereof of an effective dose of a pharmaceutical composition as defined above.

The present invention also relates to the use of a pharmaceutical composition as defined above for the manufacture of a drug, said drug being notably intended for preventing and/or treating a disorder associated with regulated necrotic cell death, such as necroptosis and/or ferroptosis. In a particular embodiment, said disorder is associated with ferroptosis, notably with both ferroptosis and necroptosis, and is as defined above.

The present invention also relates to the use, in particular a non-therapeutic use, of a pharmaceutical composition as defined above, for the in vitro preservation and/or protection of biological materials such as cells, tissues, body fluids and organs.

In the context of the present invention, “in vitro” means outside of the organism from which the biological material derives.

As used herein, the expression “preservation and/or protection of biological materials” refers to an improved survival of said biological material, allowing its conservation over time. As is clear from the present description, this improved survival is obtained by preventing ferroptosis-induced cell death in said biological materials.

Hence, the present invention also relates to the in vitro use of a pharmaceutical composition as defined above as an agent for inhibiting regulated necrotic cell death, such as necroptosis and/or ferroptosis, in a biological material.

The present invention is also directed to a method for inhibiting regulated necrotic cell death, such as necroptosis and/or ferroptosis in a biological material, which comprises exposing said biological material to a pharmaceutical composition as defined above.

In the above aspects of the present invention, the biological material is preferably a cell sample or a tissue sample.

The active ingredients of the pharmaceutical composition according to the invention, namely N-acetylcysteine or a pharmaceutical salt or a derivative thereof and the inhibitor of regulated necrotic cell death as defined above, which is in particular a ferroptosis inhibitor, preferably a ferroptosis and necroptosis inhibitor, may be administered simultaneously, separately or sequentially to a person in need thereof, in particular for inhibiting regulated necrotic cell death, such as necroptosis and/or ferroptosis, more particularly for preventing and/or treating a disorder associated with regulated necrotic cell death, such as necroptosis and/or ferroptosis.

Therefore, the present invention also relates to a pharmaceutical composition comprising:
N-acetylcysteine or a pharmaceutical salt or a derivative thereof, and
at least one inhibitor of regulated necrotic cell death, in particular at least one ferroptosis inhibitor, preferably at least one ferroptosis and necroptosis inhibitor,
   as a combination product for simultaneous, separate or sequential administration, in particular for inhibiting regulated necrotic cell death, such as necroptosis and/or ferroptosis, more particularly for preventing and/or treating a disorder associated with regulated necrotic cell death, such as necroptosis and/or ferroptosis.

The present invention also relates to a pharmaceutical composition as defined above for use as a therapeutically active ingredient in a combination or in an add-on therapeutic regimen in a person in need thereof, in particular for inhibiting regulated necrotic cell death, such as necroptosis and/or ferroptosis, more particularly for preventing and/or treating a disorder associated with regulated necrotic cell death, such as necroptosis and/or ferroptosis. Also provided is the use of a pharmaceutical composition as defined above as a therapeutically active ingredient in a combination or in an add-on therapeutic regimen in a patient in need thereof.

In some embodiments, the pharmaceutical composition of the present invention is administered simultaneously, separately or sequentially to a person in need thereof with a third active ingredient.

The pharmaceutical composition as defined above may be provided in a combination product, comprising additional products, in particular a third active ingredient, particularly intended for simultaneous, separate or sequential administration.

The third active ingredient is typically relevant for the disorder to be prevented and/or treated.

The present invention also relates to a kit comprising a pharmaceutical composition as defined above and a delivery device (a device allowing administration of said composition), in particular suitable for parenteral, enteral administration or local administration. Examples of delivery devices include but are not limited to autoinjectors, in particular multichamber syringes, transdermal patchs, pre-filled syringe or a needle free device.

### BRIEF SUMMARY OF THE FIGURES

FIG. 1 represents the maximal viability of ARPE19 cells treated with sodium iodate in the presence of NAC, compound 45 or in co-treatment.

FIG. 2 represents the maximal viability of ARPE19 cells treated with sodium iodate in the presence of NAC, compound 46 or in co-treatment.

FIG. 3 represents the maximal viability of SH-SY5Y cells treated with erastin in the presence of NAC, compound 46 or in co-treatment.

FIG. 4 represents the maximal viability of SH-SY5Y cells treated with erastin in the presence of NAC, compound 1 or in co-treatment.

FIG. 5 represents the maximal viability of HT-22 cells treated with erastin in the presence of NAC, compound 7 or in co-treatment.

FIG. 6 represents the maximal viability of HT-22 cells treated with erastin in the presence of NAC, compound 37 or in co-treatment.

FIG. 7 represents the maximal viability of HT-22 cells treated with erastin in the presence of NAC, compound 1 or in co-treatment.

FIG. 8 represents the alanine transferase (ALT) concentrations (UI/L) quantified in mouse plasma for 8 groups of mice after APAP intoxication, treated with NAC, increasing doses of compound 7 or in co-treatment.

FIGS. 9a, 9b and 9c show the synergic effect of co-treatment of NAC and compound 7 on in vivo mice model of APAP intoxication; ALT values are reported as percent of reduction of maximal ALT obtained in plasma of mice treated with 400 mg/kg APAP only; control mice (Ctrl) were mice without APAP injection.

FIG. 10 represents the maximal viability of LLC-PK1 cells treated with RSL3 in the presence of NAC, compound 7 or in co-treatment.

FIG. 11 represents the maximal viability of LLC-PK1 cells treated with RSL3 in the presence of NAC, compound 1 or in co-treatment.

FIG. 12 represents the maximal viability of LLC-PK1 cells treated with RSL3 in the presence of NAC, compound 46 or in co-treatment.

FIG. 13 represents the maximal viability of ARPE19 cells treated with sodium iodate in the presence of N-acetylcysteine amide (NACA), compound 46 or in co-treatment.

FIG. 14 represents the maximal viability of SHSY5Y cells treated with erastin in the presence of N-acetylcysteine ethyl ester, compound 7 or in co-treatment.

FIG. 15 represents the maximal viability of SHSY5Y cells treated with erastin in the presence of N-acetylcysteine ethyl ester, compound 1 or in co-treatment.

FIG. 16 represents the maximal viability of SHSY5Y cells treated with erastin in the presence of N-acetylcysteine ethyl ester, compound 46 or in co-treatment.

### EXAMPLES

The following abbreviations, commonly used in this field of art, are used in the following examples:
ALT: alanine aminotransferase
AMD: age-related macular degeneration
APAP: Acetaminophen
CI: calculation index
DMEM: Dulbecco's Modified Eagle Medium
DMSO: Dimethylsulfoxyde
H&E: haematoxylin and eosin
i.p: intraperitoneal
MTS: 3-[4,5-dimethylthiazol-2-yl]-5-[3-carboxymethoxy-phenyl]-2-[4-sulfophenyl]-2H-tetrazolium
n: number of replicates in an experiment
NAC: N-acetylcysteine
PBS: Phosphate buffered saline
SD: Standard deviation
SEM: Standard error of mean

### I. In Vitro Cellular Models of Diseases

The synergistic efficacy of the combination of NAC and regulated necrosis inhibitors was evaluated in vitro in cellular models of pathologies. Regulated necrosis is involved in several pathologies or dysfunctions of the organism and notably in liver damage linked to drug toxicity. Regulated necrosis is also involved in the pathophysiology of degenerative diseases, such as retinal degeneration, or neurodegenerative diseases such as Parkinson's disease, or in neurological disorders associated with excitotoxicity such as trauma or stroke.

### I.1. Materials and Methods

### Cell Culture

The SH-SY5Y neuronal cells (Human Neuroblastoma Cell line) and HT-22 cells (Mouse Hippocampal Neuronal Cell line) were maintained in standard DMEM with GlutaMAX medium (GIBCO), supplemented with 10% fetal bovine serum (GIBCO), at 37° C. in presence of 5% CO2.

The ARPE-19 cells (Human Retinal pigment epithelial cell line) were cultured in standard DMEM/F12 medium (GIBCO), supplemented with 10% fetal bovine serum (GIBCO), at 37° C. in presence of 5% CO2.

The LLC-PK1 cells (Pig Kidney Epithelial cells) were cultured in DMEM/F12(GIBCO), supplemented with 10% fetal bovine serum (GIBCO), at 37° C. in presence of 5% CO2.

### Cell Viability Assays

SH-SY5Y, LLC-PK1, ARPE-19 and HT22 cells were seeded in 96-well plates at a density of 10 000 or 5000 cells per well respectively, following overnight incubation. Cells were treated with 10 μM (SH-SY5Y cells) or 0.5 μM (HT22 cells) of erastin or 2 μM of RSL3 (LLCPK1) or 10 mM of sodium iodate (ARPE-19 cells) for 24 h.

Erastin and RSL3 was purchased from Selleck Chemical and sodium iodate from Sigma Aldrich. Cell viability was assessed by MTS assay (CellTiter 96@D AQueous Non-Radioactive Cell Proliferation Assay; Promega, Fitchburg, WI, USA) according to the manufacturer's instructions. This assay is based on the reduction of the 3-(4,5-dimethylthiazol-2-yl)-5-(3-carboxymethoxyphenyl)-2-(4-sulfophenyl)-2H-tetrazolium (MTS) by viable cells to form a colored formazan product. After treatment, cells were incubated 3 h at 37° C., 5% CO₂ with MTS. The absorbance was measured using a microplate reader at 490 and 630 nm and the percentage of viability was calculated by dividing the absorbance of testing compound by the absorbance of DMSO treated cells (control).

N-acetylcysteine amide (NACA) was purchased from MedChemExpress (ref: HY-110256).

N-acetylcysteine ethyl ester was purchased from MedChemExpress (ref: HY-134495).

### Analysis of Synergistic Effects of Molecules

Cells (HT-22, SHSY-5Y, LLC-PK1 and ARPE 19) were seeded in a 96-well plate in their respective medium overnight. The next day they were treated with NAC or a derivative thereof including NACA or N-acetylcysteine ethyl ester (compound A), with inhibitor compound (compound B) or with a combination of the two compounds. The analysis of the effects of the combination of the association of compound A (NAC or a derivative thereof including NACA and N-acetylcysteine ethyl ester) with another compound B is based on the independence model of Bliss. In this approach, the effect of compound alone (EA or EB) or in combination is expressed as a probability (0≤E≤1). The combined effect of the compounds is defined by:

$$EAB = EA + EB ( 1 - EA )$$

wherein EA and EB represent the effects of compound A and compound B respectively, and EAB the combined effect of the combination of A and B (expected effect on histogram). Calculation index CI is calculated as follows: CI=(EA+EB−EA*EB)/EAB
wherein CI is synergistic, antagonistic or additive when CI is under, above or equal to 1 respectively [Duarte et al., Current Research in Pharmacology and Drug Discovery, 2022].

### I.2. Model of Eye Disease

Age-related macular degeneration, or AMD, is characterized by vision loss caused by degeneration of the central cells of the retina, called the macula. Oxidative stress has been shown to play an important role in retinal cell loss through the initiation of non-apoptotic cell death, including ferroptosis [Totsuka et al., Exp. Eye Res., 2019, 181-316-324]. One model used to study retinal cell death is that of human ARPE-19 cells, a retinal pigment epithelial cell line, in the presence of sodium iodate (NaIO3, a potent oxidizing agent) [Hanus et al. Cell Death Discov. 2016, 2, 16054][Chan et al., J. Biomed. Sci., 2019, 26:40].

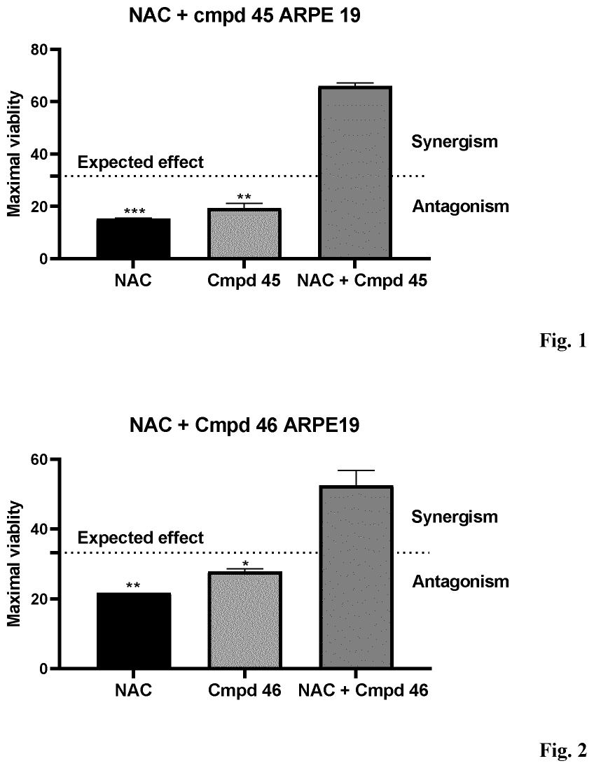

Compounds 45 (FIG. 1) and 46 (FIG. 2) were tested in this assay alone and in combination with NAC. FIG. 1 (10 μM cmpd 45; 100 μM NAC) and 2 (5 μM cmpd 46; 50 μM NAC) showed a significant effect of the tested molecules alone and a synergistic effect when combined with NAC (n=2, mean±SD, * P<0.05, ** P<0.01, *** P<0.001).

Compound 46 (FIG. 13) was tested in this assay alone and in combination with NACA (N-acetylcysteine amide). FIG. 13 (10 μM cmpd 46; 125 μM NACA) showed a significant effect of the tested molecule Compound 46 alone and a synergistic effect when combined with NACA at the tested doses (n=2, mean±SD, * P<0.05, ** P<0.01, *** P<0.001 and **** P<0.0001).

### I.3. Neurotoxicity and Excitotoxicity Models

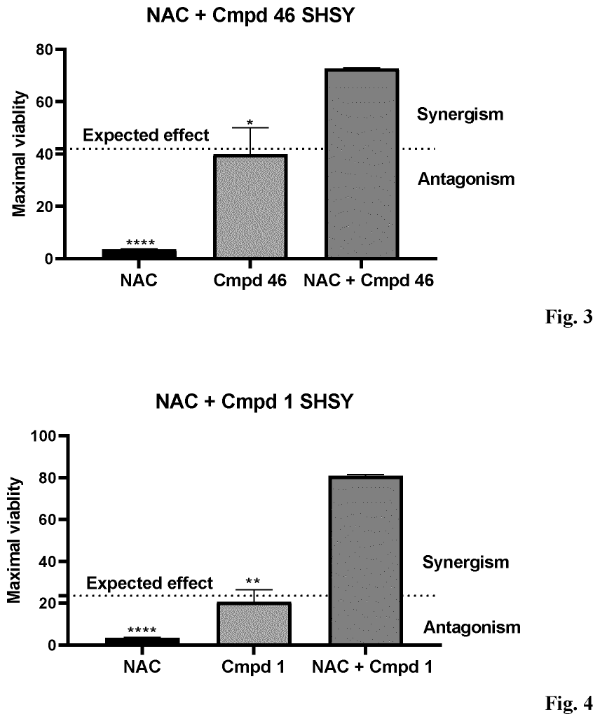

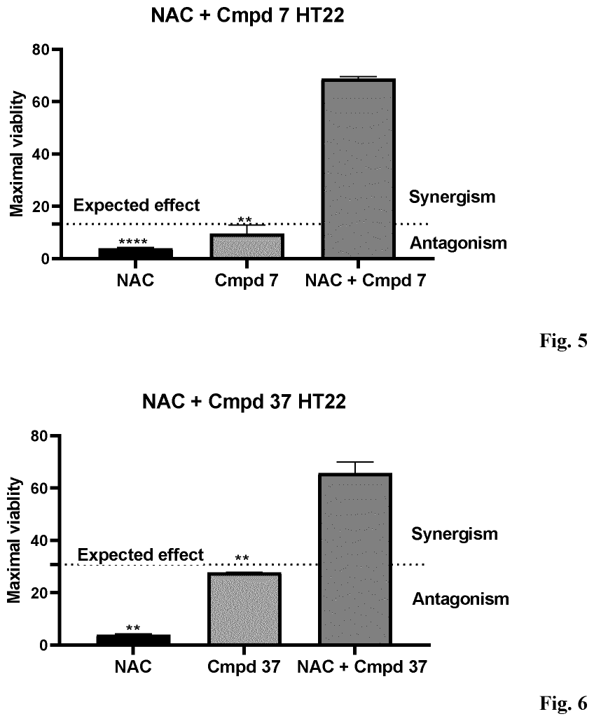

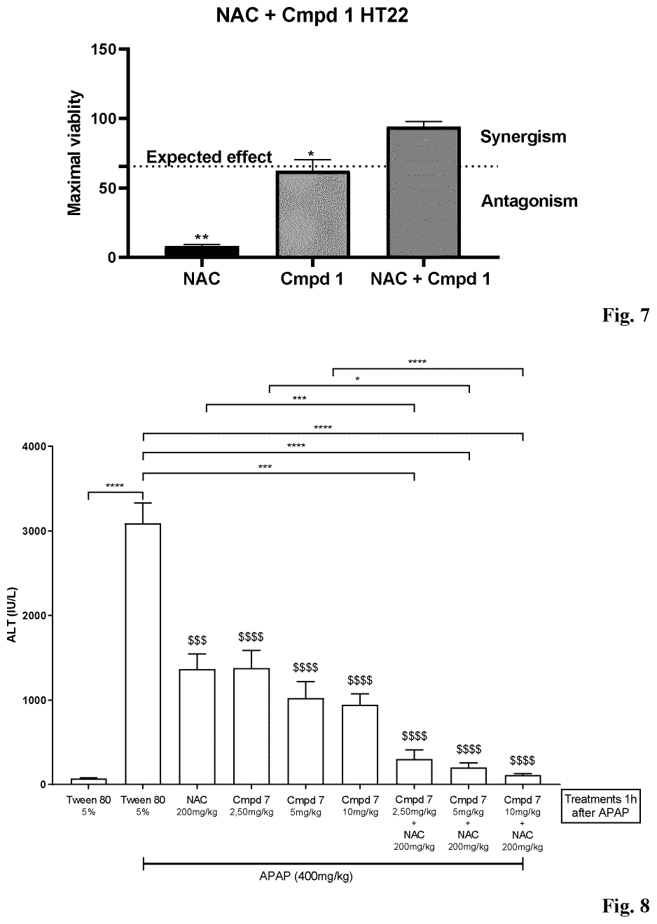

The biological activity of interest was demonstrated in two neuronal cell lines, (i) SH-SY5Y, a human neuroblastoma cell line (FIG. 3-4) and (ii) HT22, a mouse hippocampal cell line (FIG. 5-7). Cell death by ferroptosis was induced by erastin [Dixon et al., Cell, 2012, 149(5), 1060-1072] in both cell lines. Erastin is a well described ferroptosis inducer and also a molecular tool to study neuronal pathologies [Lewerenz et al., Front. Neurosci., 2018, 12: 214].

The results obtained, summarized in FIGS. 3 to 7, clearly showed the efficacy of the tested compounds on these neurotoxicity models and a synergistic efficacy when combined with NAC. Compounds 46 (FIG. 3) and 1 (FIG. 4) were added to SH-SY5Y cells in combination or not with NAC in the presence of a ferroptosis inducer (erastin). FIG. 3 (25 μM cmpd 46; 100 μM NAC) and 4 (1 μM cmpd 1; 100 μM NAC) showed a significant effect of the tested molecules alone and a synergistic effect when combined with NAC at the tested doses (n=2, mean±SD, * P<0.05, ** P<0.01, *** P<0.001 and **** P<0.0001). HT22 cells were treated with compound 7, 37 or 1 (FIGS. 5, 6 and 7 respectively) in combination or not with NAC and in the presence of a ferroptosis inducer (erastin). FIG. 5 (25 μM cmpd 7; 100 μM NAC), 6 (50 μM cmpd 37; 100 μM NAC) and 7 (5 μM cmpd 1; 100 μM NAC) showed a significant effect of the tested molecules alone and a synergistic effect when combined with NAC at the tested doses (n=2, mean±SD, * P<0.05, ** P<0.01, *** P<0.001 and **** P<0.0001).

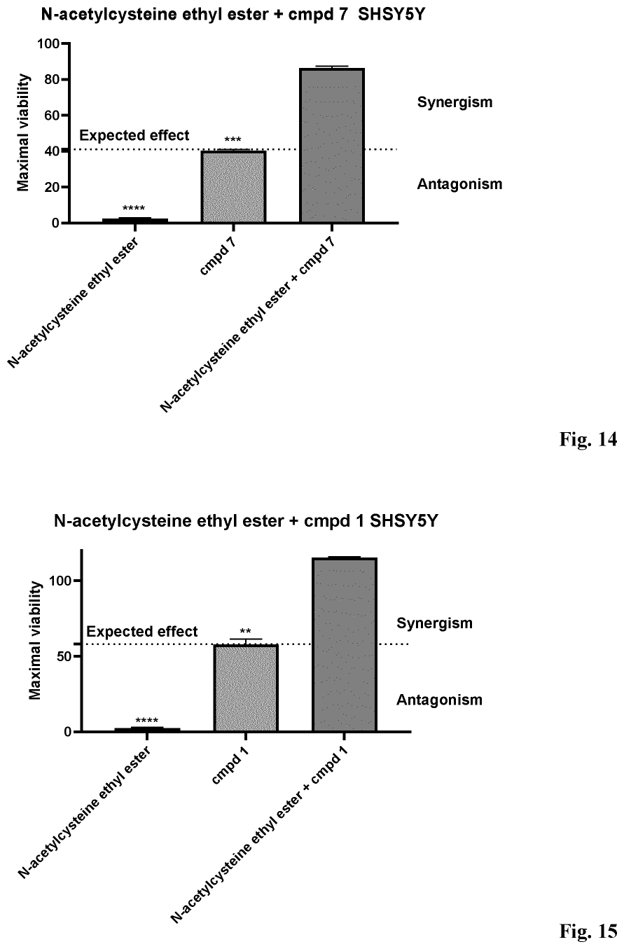

The biological activity of interest was also demonstrated with NAC derivatives in neuronal cell line SH-SY5Y. The results obtained, summarized in FIGS. 14 to 16, clearly showed the efficacy of the tested compounds on this neurotoxicity model and a synergistic efficacy when combined with N-acetylcysteine ethyl ester.

Compound 7 (FIG. 14) was added to SH-SY5Y cells in combination or not with N-acetylcysteine ethyl ester in the presence of a ferroptosis inducer (erastin). FIG. 14 (10 μM Compound 7; 100 μM N-acetylcysteine ethyl ester) showed a significant effect of the tested molecules alone and a synergistic effect when combined with NACET (n=2, mean±SD, * P<0.05, ** P<0.01, *** P<0.001).

Compound 1 (FIG. 15) was added to SH-SY5Y cells in combination or not with N-acetylcysteine ethyl ester in the presence of a ferroptosis inducer (erastin). FIG. 15 (2.5 μM Compound 1; 100 μM N-acetylcysteine ethyl ester) showed a significant effect of the tested molecules alone and a synergistic effect when combined with N-acetylcysteine ethyl ester (n=2, mean±SD, * P<0.05, ** P<0.01, *** P<0.001).

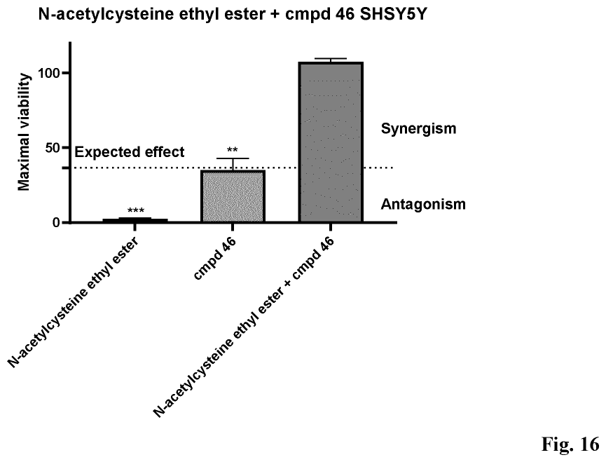

Compound 46 (FIG. 16) was added to SH-SY5Y cells in combination or not with N-acetylcysteine ethyl ester in the presence of a ferroptosis inducer (erastin). FIG. 16 (25 μM Compound 46; 100 μM N-acetylcysteine ethyl ester) showed a significant effect of the tested molecules alone and a synergistic effect when combined with N-acetylcysteine ethyl ester (n=2, mean±SD, * P<0.05, ** P<0.01, * P<0.001).

### I.4. Pig Kidney Epithelial Cells Models

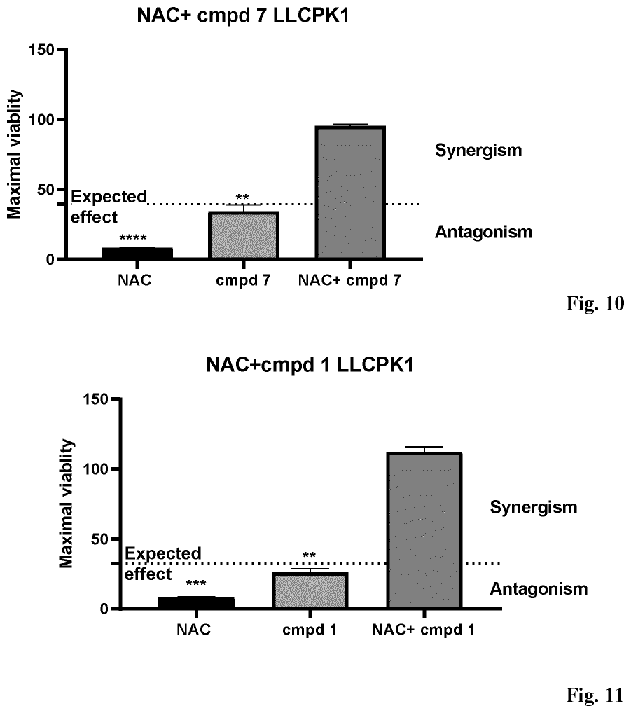

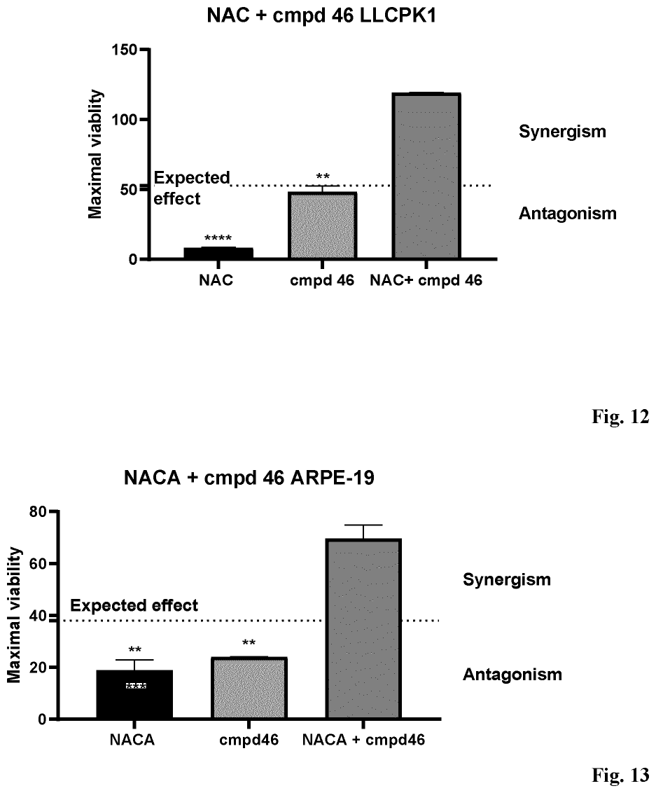

Compound 7 (FIG. 10), Compound 1 (FIG. 11) and Compound 46 (FIG. 12) were tested in this assay alone and in combination with NAC. FIG. 10 (2.5 μM Compound 7; 500 μM NAC), FIG. 11 (1 μM Compound 1; 500 μM NAC) and 12 (25 μM Compound 46; 500 μM NAC) showed a significant effect of the tested molecules alone and a synergistic effect when combined with NAC (n=2, mean±SD, * P<0.05, ** P<0.01, *** P<0.001).

### II. In Vivo Model of Pathologies

### II.1. Materials and Methods

All animal experiments were conducted in compliance with French laws and the institution's guidelines for animal welfare. This project was approved by the “Comité Régional d'Ethique et d'Expérimentation Animal” (CREAA), under the authorization APAFIS #32246-2021061616397414 v7, given by the “Ministére de l'Enseignement Supérieur de la Recherche et de l'Innovation”.

Nine-week-old C57Bl/6J male mice were purchased from Janvier Labs (Le Genest St Isle, France). After one week of acclimatization in the animal house, mice were fasted overnight before experimentation. Acetaminophen (APAP) was purchased from Sigma-Aldrich (A70-85-100 g, batch #SCLF8273) and diluted at 20 mg/mL in PBS (preheated at 45° C.). Acetaminophen intoxication was induced in mice by intraperitoneal (i.p.) injection of APAP (400 mg/kg). One hour after APAP injection, mice were treated with inhibitors by intraperitoneal injection. N-acetylcysteine (NAC, A9165-25 g, Sigma-Aldrich, batch #SLCJ1628), the reference treatment for paracetamol intoxication, was diluted at 40 mg/mL in PBS Tween80 5% and injected via intraperitoneal route at a dose of 200 mg/kg. Compound 7 was diluted at 1 mg/ml or 2 mg/ml in PBS Tween80 5% and injected by intraperitoneal injection with increasing concentrations (2.5, 5 or 10 mg/kg) or in combination with NAC (200 mg/kg). There were two groups of control mice: the first one was injected via i.p. route with PBS Tween80 5% (Tween80 5%) and the second group, treated with APAP, was administered one hour after APAP with PBS Tween80 5% (APAP-Tween80 5%) via i.p.. Mice were slaughtered eight hours after APAP injection. Blood and liver were collected.

### Biochemical Parameters

Serum alanine aminotransferase (ALT) plasma levels were measured according to the International Federation of Clinical Chemistry and Laboratory Medicine primary reference procedures using an Olympus AU2700 Autoanalyser (Olympus Optical).

### Histological Analysis

Liver fragments were fixed in 4% paraformaldehyde and embedded in paraffin. Sections of 4 μm were used for haematoxylin and eosin (H&E) staining. All paraffin-embedded liver sections were scanned with a digital slide scanner (Nanozoomer 2.0-RS, Hamamatsu Photonics, Massy, France) and files were analysed with the NDP viewer 2.5 software (Hamamatsu).

### Statistical Analysis

Results were expressed as means SEM. Mean differences between two experimental groups were assessed using the non-parametric Mann-Whitney U-test. All statistical analyses were achieved with the GraphPad Prism5 software. Calculated P values are integrated on histograms and graphs. Significance is shown as follows: $ P<0.05, $$ P<0.01, $$$ P<0.001 and $$$ P<0.0001 (comparison between APAP mice and APAP mice treated with NAC and/or compound 7); * P<0.05, ** P<0.01, *** P<0.001 and **** P<0.0001 (comparison between treated groups).

### II.2. Results

Nine Groups of Mice were Studied:
Tween80 5%, n=15
APAP (400 mg/kg)+Tween 80 5%, n=31
APAP (400 mg/kg)+NAC (200 mg/kg), n=15
APAP (400 mg/kg)+compound 7 (2.5 mg/kg), n=23
APAP (400 mg/kg)+compound 7 (5 mg/kg), n=21
APAP (400 mg/kg)+compound 7 (10 mg/kg), n=21
APAP (400 mg/kg)+compound 7 (2.5 mg/kg)+(NAC 200 mg/kg), n=9
APAP (400 mg/kg)+compound 7 (5 mg/kg)+NAC (200 mg/kg), n=9
APAP (400 mg/kg)+compound 7 (10 mg/kg)+NAC (200 mg/kg), n=13

APAP overdose induced liver injury and the release of alanine aminotransferase (ALT) in mouse plasma. Mice treated with APAP (400 mg/kg) for 8 hours showed significant increase of plasma ALT level (~3000 IU/L) in comparison with control mice only injected with Tween80 5% (~100 IU/L).

N-acetylcysteine (NAC) is an effective antidote to limit liver injury in patients with APAP intoxication. Mice treated with NAC (200 mg/kg) one hour after APAP (400 mg/kg) injection were partly protected from APAP toxicity as shown by significant reduction of ALT plasma level (~1300 IU/L) in comparison with mice treated with APAP (~3000 IU/L). Compound 7 treatment one hour after APAP was also effective to protect mice liver from APAP toxicity by reducing ALT plasma levels in a dose-dependent manner with a maximum effect observed at the dose of compound 7 (10 mg/kg) (~1000 IU/L) (FIG. 8).

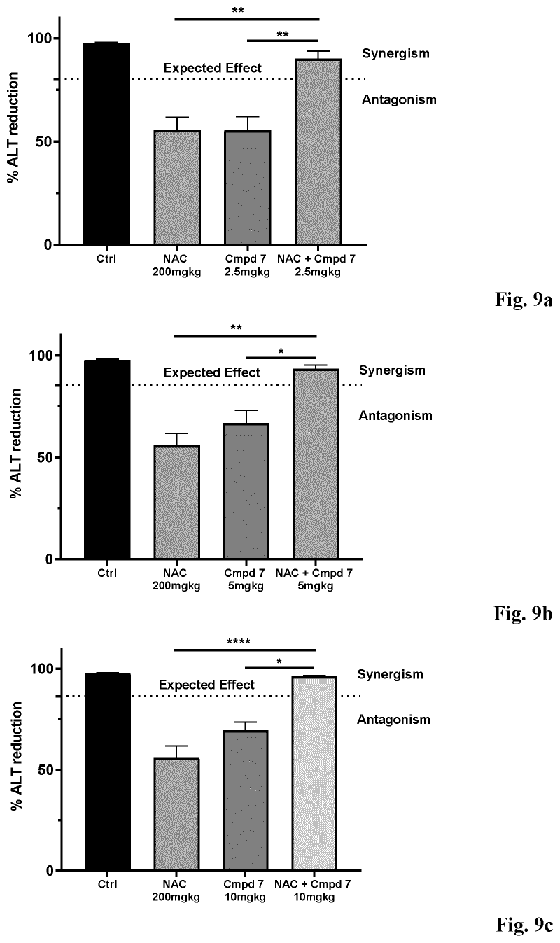

In a remarkable way, the treatment with NAC (200 mg/kg) in combination with increased concentrations of compound 7 (2.5, 5 or 10 mg/kg) had a better and synergistic protective effect against APAP toxicity than the treatment with NAC alone or compound 7 alone (FIG. 9a-c). This protective effect was dependent on the dose of compound 7 with an almost complete protection for the combination of NAC (200 mg/kg) with compound 7 (10 mg/kg) since the ALT plasma level was closed similar (~200 IU/L) to the one measured in control mice only treated with Tween80 5% (~100 IU/L) (FIG. 8).

1.-16. (canceled)

17. A pharmaceutical composition comprising a combination of N-acetylcysteine and/or a pharmaceutical salt and/or a derivative thereof with at least one inhibitor of regulated necrotic cell death.

18. The pharmaceutical composition according to claim 17, wherein the regulated necrotic cell death is a necroptosis and/or ferroptosis.

19. The pharmaceutical composition according to claim 17, wherein the inhibitor of regulated necrotic cell death is a ferroptosis inhibitor.

20. The pharmaceutical composition of claim 17, wherein the inhibitor of regulated necrotic cell death is:
(A) a compound of the following general formula (I): 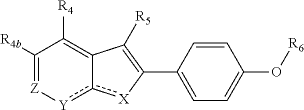
or a pharmaceutically acceptable salt and/or solvate thereof,
wherein: 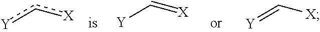
when 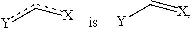
X is N, Y is N(R₂) and Z is C(H);
(ii) when 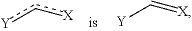
X is N(R₁), and
Y is N or N⁺(O⁻) and Z is C(R₃), or
Y is CH and Z is N, or
Y and Z are CH;
and wherein:
R₁ and R₂ represent, independently of each other, a hydrogen atom, CN, NO₂, OR₇, SR₈, NR₉R₁₀, C(O)R₁₁, CO₂R₁₂, OC(O)R₁₃, NR₁₄C(O)R₁₅, C(O)NR₁₆R₁₇, S(O)Rₛ, SO₂Rₛ′, a (C₁-C₆)alkyl, a (C₁-C₆)haloalkyl, a —(C₁-C₆)alkyl-[O—(C₁-C₆)alkyl]ₘ—NR_{N1}R_{N2} group with m ranging from 1 to 6, an aryl, an aryl-(C₁-C₆)alkyl, a heterocyclyl, or a heterocyclyl-(C₁-C₆)alkyl group, wherein said aryl or heterocyclyl group is optionally substituted by one or more substituents selected from the group consisting of a halogen atom, CN, NO₂, OR₁₈, SR₁₉, NR₂₀R₂₁, C(O)R₂₂, CO₂R₂₃, OC(O)R₂₄, NR₂₆C(O)R₂₆, C(O)NR₂₇R₂₈, a (C₁-C₆)alkyl and a (C₁-C₆)haloalkyl group;
R₃, R₄, R_{4b} and R₅ represent, independently of each other, a hydrogen atom, a halogen atom, CN, OR₂₉, SR₃₀, NR₃₁R₃₂, C(O)R₃₃, CO₂R₃₄, OC(O)R₃₅, NR₃₆C(O)R₃₇, C(O)NR₃₈R₃₉, a (C₁-C₆)alkyl, a (C₁-C₆)haloalkyl group, said alkyl or haloalkyl group being optionally substituted by one or more substituents selected from the group consisting of OR₄₀, SR₄₁ and NR₄₂R₄₃, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, CN, NO₂, OR₄₄, SR₄₅, NR₄₆R₄₇, C(O)R₄₈, CO₂R₄₉, OC(O)R₅₀, NR₅₁C(O)R₅₂, C(O)NR₅₃R₅₄, a (C₁-C₆)alkyl and a (C₁-C₆)haloalkyl group;
R₆ represents a hydrogen atom, a (C₁-C₆)alkyl, an aryl-(C₁-C₆)alkyl, a heterocyclyl-(C₁-C₆)alkyl, a —(C₁-C₆)alkyl-[O—(C₁-C₆)alkyl]ₘ-—NR_{N′1}R_{N′2} group with m′ ranging from 1 to 6 or a (C₁-C₆)alkylcarbonyl group, said (C₁-C₆)alkyl, aryl-(C₁-C₆)alkyl, and (C₁-C₆)alkylcarbonyl group being optionally substituted with one or more substituents selected from the group consisting of OH, SH, NH₂, a (C₁-C₆)alkoxy, a (C₁-C₆)thioalkoxy, a (C₁-C₆)alkylamino and a di((C₁-C₆)alkyl)amino group;
Rₛ and Rₛ′ represent, independently of each other a (C₁-C₆)alkyl, an aryl or an aryl-(C₁-C₆)alkyl group;
R₇-R₁₀, R₁₂, R₁₄ and R₁₆-R₁₇ represent, independently of each other, a hydrogen atom, a (C₁-C₆)alkyl, an aryl or an aryl-(C₁-C₆)alkyl group;
R₁₁, R₁₃ and R₁₅ represent, independently of each other, a hydrogen atom, a (C₁-C₆)alkyl, an aryl, an aryl-(C₁-C₆)alkyl, a (C₁-C₆)alkoxy, a (C₁-C₆)alkylamino or a di((C₁-C₆)alkyl)amino group;
R₁₈ to R₂₈ represent, independently of each other, a hydrogen atom, a (C₁-C₆)alkyl or an aryl group;
R₂₉ to R₃₉ represent, independently of each other, a hydrogen atom, a (C₁-C₆)alkyl, heterocyclyl-(C₁-C₆)alkyl, an aryl or an aryl-(C₁-C₆)alkyl group, said aryl group being optionally substituted by one or more substituents selected from the group consisting of a halogen atom, CN, NO₂, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group;
R₄₀ to R₄₃ represent, independently of each other, a hydrogen atom or a (C₁-C₆)alkyl group;
R₄₄ to R₅₄ represent, independently of each other, a hydrogen atom, a (C₁-C₆)alkyl or an aryl group;
R₅₅ to R₆₅ represent, independently of each other, a hydrogen atom, a (C₁-C₆)alkyl, aryl-(C₁-C₆)alkyl group or an aryl group; and
R_{N1}, R_{N′1}, R_{N2} and R_{N′2} represent, independently of each other, a hydrogen atom, a (C₁-C₆)alkyl group, an aryl-(C₁-C₆)alkyl group or an aryl group; or
(B) a compound of the following general formula (II): 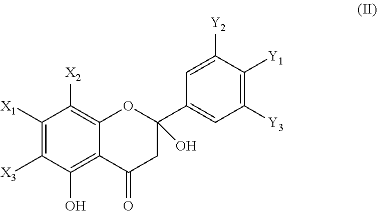
or a pharmaceutically acceptable salt and/or solvate thereof, wherein:
X₁, X₂ and X₃ represent, independently of each other, a hydrogen atom, a (C₁-C₆)alkyl, an aryl, an aryl-(C₁-C₆)alkyl or an OH group, or a group selected from ORₓ, SRₓ, SO₂Rₓ and NRₓR_{z},
wherein at least one of X₁, X₂ and X₃ represents a (C₁-C₆)alkyl, an aryl or an aryl-(C₁-C₆)alkyl group, or a group selected from ORₓ, SRₓ, SO₂Rₓ and NRₓR_{z}, wherein
Rₓ a (C₁-C₆)alkyl, an aryl or an aryl-(C₁-C₆)alkyl group,
R_{z} is a hydrogen atom or a (C₁-C₆)alkyl group, and
the aryl groups are optionally substituted with one or several groups selected from a halogen atom, —OR₆₆, —NR₆₇R₆₈, —SR₆₉, —S(O)R₇₀, —SO₂R₇₁, —OCOR₇₂, —CO₂R₇₃, —CONR₇₄R₇₅, —CO₂R₇₆, nitro (—NO₂) and cyano (—CN);
Y₁, Y₂ and Y₃ represent, independently of each other, a hydrogen atom, a (C₁-C₆)alkyl, an aryl, an aryl-(C₁-C₆)alkyl or an OH group, or a group selected from OR_{y}, SR_{y}, SO₂R_{y} and NR_{y}R′_{z},
wherein at least one of Y₁, Y₂ and Y₃ represents a (C₁-C₆)alkyl, an aryl or an aryl-(C₁-C₆)alkyl group, or a group selected from OR_{y}, SR_{y}, SO₂R_{y} and NR_{y}R′_{z}, wherein
R_{y} is a (C₁-C₆)alkyl, an aryl or an aryl-(C₁-C₆)alkyl group,
R′_{z} is a hydrogen atom or a (C₁-C₆)alkyl group, and
the aryl groups are optionally substituted with one or several groups selected from a halogen atom, —OR₆₆, —NR₆₇R₆₈, —SR₆₉, —S(O)R₇₀, —SO₂R₇₁, —OCOR₇₂, —CO₂R₇₃, —CONR₇₄R₇₅, —CO₂R₇₆, nitro (—NO₂) and cyano (—CN); and
R₆₆ to R₇₇ are, independently of one another, a hydrogen atom or a (C₁-C₆)alkyl group.

21. The pharmaceutical composition of claim 20, wherein the inhibitor of regulated necrotic cell death is a compound of formula (I), wherein:
R₁ and R₂ represent, independently of each other, a hydrogen atom, CN, NO₂, OR₇, SR₈, NR₉R₁₀, C(O)R₁₁, CO₂R₁₂, OC(O)R₁₃, NR₁₄C(O)R₁₅, C(O)NR₁₆R₁₇, S(O)Rₛ, SO₂Rₛ′, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, CN, NO₂, OR₁₈, SR₁₉, NR₂₀R₂₁, C(O)R₂₂, CO₂R₂₃, OC(O)R₂₄, NR₂₅C(O)R₂₆, C(O)NR₂₇R₂₈, a (C₁-C₆)alkyl and a (C₁-C₆)haloalkyl group;
R₃, R₄, R_{4b} and R₅ represent, independently of each other, a hydrogen atom, a halogen atom, CN, OR₂₉, SR₃₀, NR₃₁R₃₂, C(O)R₃₃, CO₂R₃₄, OC(O)R₃₅, NR₃₆C(O)R₃₇, C(O)NR₃₈R₃₉, a (C₁-C₆)alkyl, a (C₁-C₆)haloalkyl group, said alkyl or haloalkyl group being optionally substituted by one or more substituents selected from the group consisting of OR₄₀, SR₄₁ and NR₄₂R₄₃, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, CN, NO₂, OR₄₄, SR₄₅, NR₄₆R₄₇, C(O)R₄₈, CO₂R₄₉, OC(O)R₅₀, NR₅₁C(O)R₅₂, C(O)NR₅₃R₅₄, a (C₁-C₆)alkyl and a (C₁-C₆)haloalkyl group;
R₆ represents a hydrogen atom, a (C₁-C₆)alkyl, an aryl-(C₁-C₆)alkyl or a —CH₂—CH₂—O—CH₂—CH₂—NH₂ group, or a (C₁-C₆)alkylcarbonyl group optionally substituted with one or more substituents selected from the group consisting of OH, SH, NH₂, a (C₁-C₆)alkoxy, a (C₁-C₆)thioalkoxy, a (C₁-C₆)alkylamino and a di((C₁-C₆)alkyl)amino group;
Rₛ and Rₛ′ represent, independently of each other a (C₁-C₆)alkyl, an aryl or an aryl-(C₁-C₆)alkyl group;
R₇-R₁₀, R₁₂, R₁₄ and R₁₆-R₁₇ represent, independently of each other, a hydrogen atom, a (C₁-C₆)alkyl, an aryl or an aryl-(C₁-C₆)alkyl group;
R₁₁, R₁₃ and R₁₅ represent, independently of each other, a hydrogen atom, a (C₁-C₆)alkyl, an aryl, an aryl-(C₁-C₆)alkyl, a (C₁-C₆)alkoxy, a (C₁-C₆)alkylamino or a di((C₁-C₆)alkyl)amino group;
R₁₈ to R₂₈ represent, independently of each other, a hydrogen atom, a (C₁-C₆)alkyl or an aryl group;
R₂₉ to R₃₉ represent, independently of each other, a hydrogen atom, a (C₁-C₆)alkyl, an aryl or an aryl-(C₁-C₆)alkyl group, said aryl group being optionally substituted by one or more substituents selected from the group consisting of a halogen atom, CN, NO₂, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group;
R₄₀ to R₄₃ represent, independently of each other, a hydrogen atom or a (C₁-C₆)alkyl group;
R₄₄ to R₅₄ represent, independently of each other, a hydrogen atom, a (C₁-C₆)alkyl or an aryl group; and
R₅₅ to R₆₅ represent, independently of each other, a hydrogen atom, a (C₁-C₆)alkyl or an aryl group.

22. The pharmaceutical composition of claim 20, wherein the inhibitor of regulated necrotic cell death is a compound of formula (I), wherein:
R₁ represents a hydrogen atom, CN, NO₂, OR₇, SR₈, NR₉R₁₀, C(O)R₁₁, CO₂R₁₂, OC(O)R₁₃, NR₁₄C(O)R₁₅, C(O)NR₁₆R₁₇, S(O)Rₛ, SO₂Rₛ′, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group is optionally substituted by one substituent selected from the group consisting of a halogen atom, CN, NO₂, OR₁₈, SR₁₉, NR₂₀R₂₁, a (C₁-C₆)alkyl and a (C₁-C₆)haloalkyl group, wherein R₁₈ to R₂₁ represent, independently of each other, a hydrogen atom or a (C₁-C₆)alkyl group and Rₛ and Rₛ′ represent, independently of each other a (C₁-C₆)alkyl or an aryl group, preferably an aryl group,
preferably, R₁ represents a hydrogen atom, CN, OR₇, C(O)R₁₁, CO₂R₁₂, OC(O)R₁₃, SO₂Rₛ′, a (C₁-C₆)alkyl, a heterocyclyl or a heterocyclyl-(C₁-C₆)alkyl group, wherein said heterocyclyl group is optionally substituted by NO₂,
more preferably, R₁ represents a hydrogen atom or a (C₁-C₆)alkyl group, in particular a hydrogen atom or a (C₁-C₃)alkyl group, advantageously a hydrogen atom;
R₂ represents C(O)R₁₁, CO₂R₁₂, C(O)NR₁₆R₁₇, a (C₁-C₆)alkyl, a (C₁-C₆)haloalkyl, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom OR₁₈, SR₁₉, NR₂₀R₂₁, a (C₁-C₆)alkyl and a (C₁-C₆)haloalkyl group, wherein R₁₈ to R₂₁ represent, independently of each other, a hydrogen atom or a (C₁-C₆)alkyl group,
preferably R₂ represents CO₂R₁₂, C(O)NR₁₆R₁₇ or an aryl-(C₁-C₆)alkyl group, wherein said aryl group is optionally substituted by one or more substituents selected from the group consisting of a halogen atom, OR₁₈, SR₁₉ and NR₂₀R₂₁, notably OR₁₈;
R₃ represents a hydrogen atom, a halogen atom, a (C₁-C₆)alkyl group, CN, OR₂₉, SR₃₀, NR₃₁R₃₂, C(O)R₃₃, CO₂R₃₄, OC(O)R₃₅, NR₃₆C(O)R₃₇, C(O)NR₃₈R₃₉, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, OR₄₄, SR₄₅, NR₄₆R₄₇, a (C₁-C₆)alkyl and a (C₁-C₆)haloalkyl group, wherein R₂₉ to R₃₇ represent, independently of each other, a hydrogen atom or a (C₁-C₆)alkyl group, and R₄₄ to R₄₇ represent, independently of each other, a hydrogen atom or a (C₁-C₆)alkyl group,
preferably R₃ represents a hydrogen atom, a halogen atom, a (C₁-C₆)alkyl group, CN, OR₂₉, SR₃₀, NR₃₁R₃₂, OC(O)R₃₅, NR₃₆C(O)R₃₇, a heterocyclyl or a heterocyclyl-(C₁-C₆)alkyl group, wherein said heterocyclyl group is optionally substituted by one or more substituents selected from the group consisting of a halogen atom, OR₄₄, SR₄₅, NR₄₆R₄₇ and a (C₁-C₆)alkyl a group,
more preferably R₃ represents a hydrogen atom, a halogen atom, a (C₁-C₆)alkyl group, CN, NR₃₁R₃₂, OC(O)R₃₆, a heterocyclyl or a heterocyclyl-(C₁-C₆)alkyl group, wherein said heterocyclyl group is optionally substituted by one or more substituents selected from the group consisting of a OR₄₄, SR₄₅ and NR₄₆R₄₇, notably OR₄₄,
even more preferably R₃ represents a hydrogen atom, a halogen atom or a (C₁-C₃)alkyl group, advantageously a hydrogen atom or a halogen atom;
R₄ represents a hydrogen atom, a halogen atom, CN, OR₂₉, SR₃₀, NR₃₁R₃₂, a (C₁-C₆)alkyl, a (C₁-C₆)haloalkyl group, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, CN, NO₂, OR₄₄, SR₄₅, NR₄₆R₄₇, C(O)₂R₄₈, CO₂R₄₉, OC(O)R₅₀, NR₅₁C(O)R₅₂, C(O)NR₅₃R₅₄, a (C₁-C₆)alkyl and a (C₁-C₆)haloalkyl group,
preferably R₄ represents a hydrogen atom, a halogen atom, OR₂₉, SR₃₀, NR₃₁R₃₂, a (C₁-C₆)alkyl, an aryl or a heterocyclyl group, wherein said aryl or heterocyclyl group is optionally substituted by one or more substituents selected from the group consisting of a halogen atom, OR₄₄, SR₄₅, NR₄₆R₄₇, C(O)R₄₈, CO₂R₄₉, C(O)NR₅₃R₅₄, a (C₁-C₆)alkyl and a (C₁-C₆)haloalkyl group,
more preferably R₄ represents a hydrogen atom, a halogen atom, NR₃₁R₃₂, a (C₁-C₆)alkyl, an aryl or a heterocyclyl group, wherein said aryl or heterocyclyl group is optionally substituted by one or more substituents selected from the group consisting of C(O)R₄₈ and a (C₁-C₆)alkyl group,
even more preferably R₄ represents a hydrogen atom or a (C₁-C₃)alkyl group, advantageously a hydrogen atom,
R_{4b} represents a hydrogen atom, a halogen atom, a (C₁-C₆)alkyl group, OR₂₉ or NR₃₁R₃₂, preferably a hydrogen atom, a halogen atom, a (C₁-C₃)alkyl group or OR₂₉, more preferably a hydrogen atom or a (C₁-C₃)alkyl group, advantageously a hydrogen atom,
wherein, in the above definitions of R₄ and R_{4b}, R₂₉ to R₃₂ represent, independently of each other, a hydrogen atom, a (C₁-C₆)alkyl, an aryl or an aryl-(C₁-C₆)alkyl group, said aryl group being optionally substituted by one or more substituents selected from the group consisting of a halogen atom, OR₅₅, SR₅₆, NR₅₇R₅₈, C(O)R₅₉, CO₂R₆₀, OC(O)R₆₁, NR₆₂C(O)R₆₃, C(O)NR₆₄R₆₅, a (C₁-C₆)alkyl and a (C₁-C₆)haloalkyl group, notably C(O)R₅₉, CO₂R₆₀, C(O)NR₆₄R₆₅, a (C₁-C₆)alkyl and a (C₁-C₆)haloalkyl group, in particular C(O)R₅₉, R₄₈ represents a hydrogen atom, a (C₁-C₆)alkyl or an aryl group, notably an aryl group, and R₅₅ to R₆₅ represent, independently of each other, a hydrogen atom, a (C₁-C₆)alkyl or an aryl group, notably an aryl group;
R₅ represents a hydrogen atom, a halogen atom, CN, OR₂₉, SR₃₀, NR₃₁R₃₂, a (C₁-C₆)alkyl, a (C₁-C₆)haloalkyl group, said alkyl or haloalkyl group being optionally substituted by one or more substituents selected from the group consisting of OR₄₀, SR₄₁ and NR₄₂R₄₃, an aryl, a heterocyclyl, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, OR₄₄, SR₄₅, NR₄₆R₄₇, a (C₁-C₆)alkyl and a (C₁-C₆)haloalkyl group, wherein R₂₉ to R₃₂ represent, independently of each other, a hydrogen atom or a (C₁-C₆)alkyl group, and R₄₀ to R₄₃ represent, independently of each other, a hydrogen atom or a (C₁-C₆)alkyl group, notably a hydrogen atom,
preferably R₅ represents a hydrogen atom, a halogen atom, a (C₁-C₆)alkyl, a (C₁-C₆)haloalkyl group, said alkyl or haloalkyl group being optionally substituted by one or more substituents selected from the group consisting of OR₄₀, SR₄₁ and NR₄₂R₄₃, an aryl-(C₁-C₆)alkyl or a heterocyclyl-(C₁-C₆)alkyl group, wherein said aryl or heterocyclyl group is optionally substituted by one or more substituents selected from the group consisting of a halogen atom, a (C₁-C₆)alkyl and a (C₁-C₆)haloalkyl group,
more preferably, Re represents a hydrogen atom, a halogen atom, a (C₁-C₆)alkyl, a (C₁-C₆)haloalkyl group or a heterocyclyl-(C₁-C₆)alkyl group, said alkyl or haloalkyl group being optionally substituted by OR₄₀, and said heterocyclyl being optionally substituted by one or more (C₁-C₆)alkyl group,
even more preferably, R₅ represents a hydrogen atom or a (C₁-C₃)alkyl group, advantageously a hydrogen atom; and
R₆ represents a hydrogen atom, a (C₁-C₃)alkyl, an aryl-(C₁-C₃)alkyl or a —CH₂—CH₂—O—CH₂—CH₂—NH₂ group, or a (C₁-C₆)alkylcarbonyl group optionally substituted with one or more substituents selected from the group consisting of OH, SH, NH₂, a (C₁-C₃)alkoxy, a (C₁-C₃)thioalkoxy and a (C₁-C₃)alkylamino group,
preferably Re represents a hydrogen atom, a methyl, an ethyl, a benzyl, a —CH₂—CH₂—O—CH₂—CH₂—NH₂ or a (C₁-C₆)alkylcarbonyl group optionally substituted with one or more substituents selected from the group consisting of OH, NH₂ and a thiomethyl group, in particular R₆ represents a hydrogen atom or a —CH₂—CH₂—O—CH₂—CH₂—NH₂ group.

23. The pharmaceutical composition of claim 20, wherein the inhibitor of regulated necrotic cell death is a compound of formula (II), wherein:
X₁ represents a (C₁-C₆)alkyl, an aryl, an aryl-(C₁-C₆)alkyl or an ORₓ group, wherein Rₓ is a (C₁-C₆)alkyl, an aryl or an aryl-(C₁-C₆)alkyl group;
X₂ and Xs₃ represent, independently of each other, a hydrogen atom or a (C₁-C₆)alkyl group; and
Y₁, Y₂ and Y₃ represent, independently of each other, a hydrogen atom, a (C₁-C₆)alkyl, an aryl, an aryl-(C₁-C₆)alkyl, an OH or an OR_{y} group, wherein at least one of Y₁, Y₂ and Y₃ represents a (C₁-C₆)alkyl, an aryl, an aryl-(C₁-C₆)alkyl or an OR_{y} group, wherein R_{y} is selected from a (C₁-C₆)alkyl, an aryl and an aryl-(C₁-C₆)alkyl group.

24. The pharmaceutical composition of claim 23, wherein:
X₁ represents an ORₓ group, Rₓ being advantageously a (C₁-C₆)alkyl group;
Y₁ represents an OR_{y} group, R_{y} being advantageously an aryl-(C₁-C₆)alkyl group, and Y₂ and Y₃ each represent a hydrogen atom.

25. The pharmaceutical composition according to claim 20, wherein the inhibitor of regulated necrotic cell death is:
(a) a compound of the following general formula (I.iii): 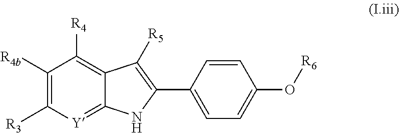
or a pharmaceutically acceptable salt and/or solvate thereof, wherein:
R₃, R₄, R_{4b} and R₅ represent, independently of each other, a hydrogen atom, a halogen atom or a (C₁-C₆)alkyl group, in particular a hydrogen atom, a halogen atom or a (C₁-C₃)alkyl group, preferably a hydrogen atom or a halogen atom, and
R₆ represents a hydrogen atom, a (C₁-C₃)alkyl or a —CH₂—CH₂—O—CH₂—CH₂—NH₂ group, in particular a hydrogen atom or a —CH₂—CH₂—O—CH₂—CH₂—NH₂ group;
or
(b) a compound of the following general formula (II.i): 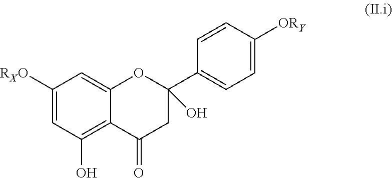
or a pharmaceutically acceptable salt and/or solvate thereof, wherein:
Rₓ represents a (C₁-C₆)alkyl group, notably a (C₁-C₃)alkyl group such as methyl, ethyl, n-propyl, more advantageously methyl, and
R_{y} represents an aryl-(C₁-C₆)alkyl group, such as benzyl or —CH₃-naphtyl, more preferably benzyl.

26. The pharmaceutical composition of claim 17, wherein the inhibitor of regulated necrotic cell death is selected from the group consisting of: 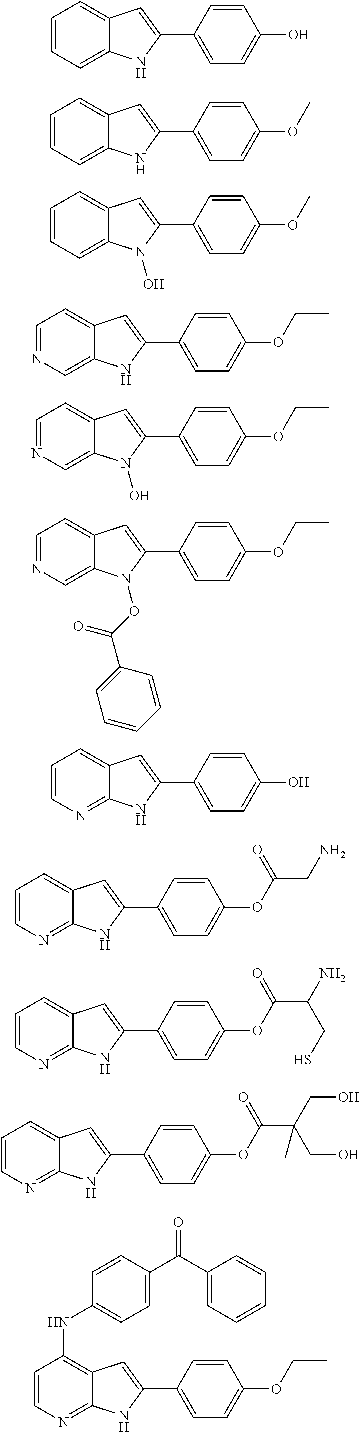 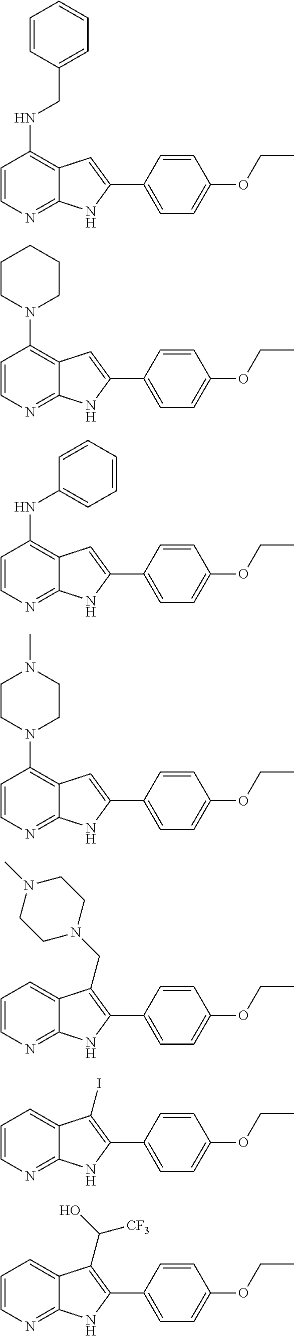 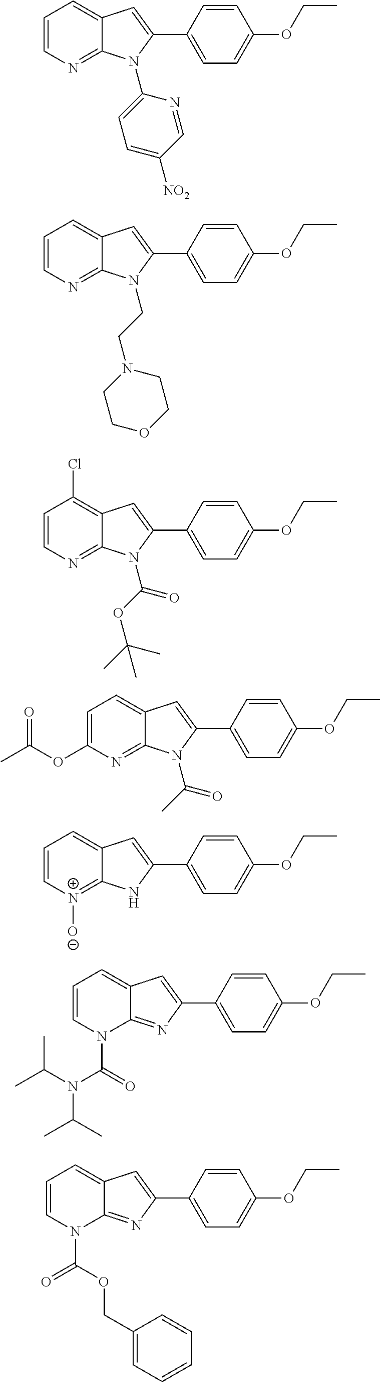 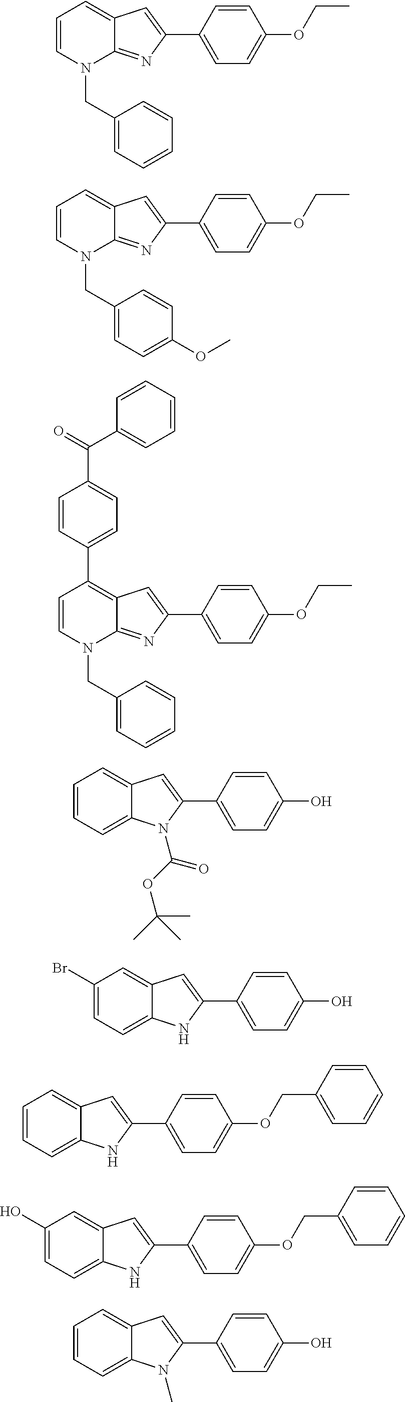 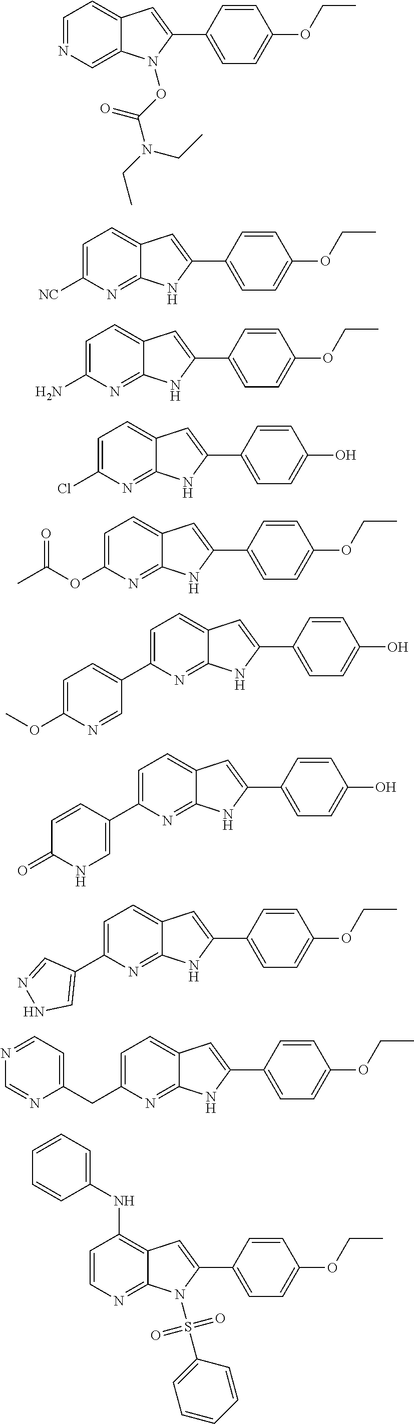 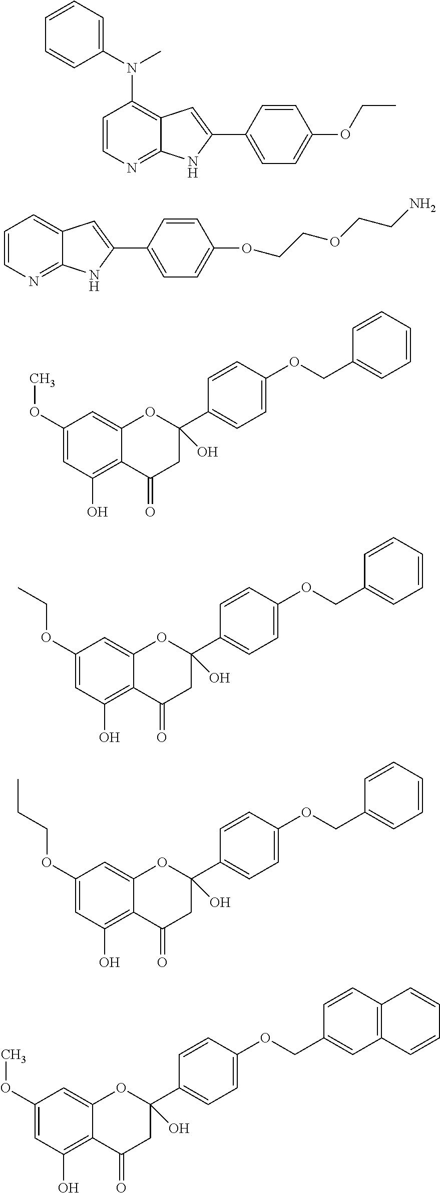
and the pharmaceutically acceptable salts and/or solvates thereof.

27. The pharmaceutical composition of claim 17, wherein the inhibitor of regulated necrotic cell death is selected from the group consisting of: 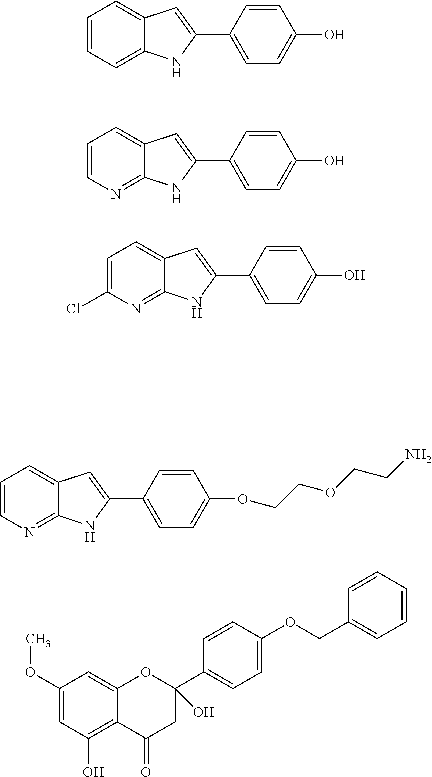 [SMILES: Oc1ccc(-c2cc3ccccc3[nH]2)cc1 ; Oc1ccc(-c2cc3cccnc3[nH]2)cc1 ; Oc1ccc(-c2cc3ccc(Cl)nc3[nH]2)cc1 ; NCCOCCOc1ccc(-c2cc3cccnc3[nH]2)cc1 ; COc1cc(O)c2c(c1)OC(O)(c1ccc(OCc3ccccc3)cc1)CC2=O]
and the pharmaceutically acceptable salts and/or solvates thereof.

28. The pharmaceutical composition of claim 17, wherein the derivative of N-acetylcysteine is N-acetylcysteine amide, and/or N-acetylcysteine ethyl ester and/or N-acetylcysteine methyl ester, and/or salt(s) thereof.

29. A pharmaceutical composition of claim 17 comprising:
N-acetylcysteine and/or a pharmaceutical salt and/or a derivative thereof, and
at least one inhibitor of regulated necrotic cell death
as a combination product for simultaneous, separate, or sequential administration.

30. The pharmaceutical composition of claim 29, wherein the derivative of N-acetylcysteine is N-acetylcysteine amide, and/or N-acetylcysteine ethyl ester, and/or N-acetylcysteine methyl ester, and/or salt(s) thereof.

31. A method for preventing and/or treating a disorder associated with regulated necrotic cell death, such as necroptosis and/or ferroptosis, comprising administering to a person in need thereof a therapeutically effective dose of the pharmaceutical composition of claim 17.

32. The method of claim 31, wherein the disorder is associated with ferroptosis.

33. The method of claim 32, wherein the disorder is selected from the group consisting of brain diseases or disorders including neurodegenerative diseases or disorders, stroke, traumatic brain injury, epilepsy; eye diseases or disorders including retinopathy, degenerative eye diseases or disorders; infectious diseases; autoimmune diseases; inflammatory diseases; pathologies related to stress-induced premature tissue senescence; liver injury including acute liver failure and chronic liver diseases; hypertension; haemochromatosis; hemolytic disorders; ischemic disorders affecting the heart, brain, or kidneys; kidney injury including acute kidney injury, renal ischemia-reperfusion injury, acute tubular necrosis, liver fibrosis; heart injury; aortic aneurysm; pancreatitis; radiation-induced necrosis; chronic obstructive pulmonary disease, acute respiratory distress disorder; diseases related to transplantation; and cancers including liver cancers, eye cancers, brain cancers, kidney cancers.

34. A method for the in vitro preservation and/or protection of biological materials such as cells, tissues, body fluids and organs, comprising using a pharmaceutical composition of claim 17 as an agent for inhibiting regulated necrotic cell death.
